(19) United States
(12) Patent Application Publication
(10) Pub. No.: US 2024/0116231 A1
(43) Pub. Date: Apr. 11, 2024

(54) IMPLANTABLE MEDICAL DEVICES AT LEAST PARTIALLY FORMED FROM A THERMOSET MATERIAL

(71) Applicant: INSPIRE MEDICAL SYSTEMS, INC., Golden Valley, MN (US)

(72) Inventors: Kevin Verzal, Lino Lakes, MN (US); John Rondoni, Plymouth, MN (US); Nathan Olson, Golden Valley, MN (US)

(73) Assignee: INSPIRE MEDICAL SYSTEMS, INC., Golden Valley, MN (US)

(21) Appl. No.: 18/275,277

(22) PCT Filed: Jan. 31, 2022
(86) PCT No.: PCT/US2022/014501

§ 371 Date: Aug. 1, 2023

Related U.S. Application Data
(60) Provisional application No. 63/145,705, filed on Feb. 4, 2021.

(51) Int. Cl. B29C 48/18; A61N 1/05; B29C 48/00; B29C 48/03

(52) U.S. Cl. CPC B29C 48/18; A61N 1/0556; B29C 48/002; B29C 48/0021; B29C 48/03; B29L 2031/753

(57) ABSTRACT

Examples are directed to a system, device and/or method comprising forming at least one lead portion of a medical lead body in a non-linear configuration from a thermoset material. Some examples are directed to a system, device and/or method comprising forming a body supporting at least one contact electrode, the body at least partially defining a re-closable lumen and comprising engaging portions, at least one of the engaging portions being formed from a silicone material.

### BACKGROUND

Many patients benefit from therapy provided by an implantable medical device (IMD). For example, a portion of the population suffers from various forms of sleep disorder breathing (SDB). In some patients, external breathing therapy devices and/or surgical interventions may fail to treat the SDB behavior. Such IMD devices may be formed of a variety of material.

### BRIEF DESCRIPTION OF THE DRAWINGS

FIG. 1 is a flow diagram schematically representing an example method comprising forming at least one lead portion in a non-linear configuration.

FIGS. 2A-2F illustrate example devices comprising at least one lead portion of a medical lead body in a non-linear configuration.

FIGS. 3A-3B illustrate side views of two different example medical lead bodies comprising at least two lead portions.

FIG. 4 is a block diagram schematically representing an example IMD.

FIGS. 5A-5E are diagrams schematically representing deployment of example IMDs, which comprise at least one lead portion of a medical lead body in a non-linear configuration.

FIGS. 6A-6C are diagrams which may comprise part of and/or are example implementations of a flow diagram in example methods.

FIGS. 7A-12E are diagrams, which may comprise part of and/or are example implementations of a flow diagram in example methods.

FIG. 13 is a flow diagram schematically representing an example method comprising forming a body supporting at least one contact electrode formed at least partially from a silicone material.

FIGS. 14A-14C are flow diagrams, which may comprise part of and/or are example implementations of a flow diagram in example methods.

FIGS. 15-19 are diagrams schematically illustrating different example devices comprising a body formed at least partially from a silicone material.

FIGS. 20A-26C are diagrams which may comprise part of and/or are example implementations of a flow diagram in example methods.

### DETAILED DESCRIPTION

In the following detailed description, reference is made to the accompanying drawings which form a part hereof, and in which is shown by way of illustration specific examples in which the disclosure may be practiced. It is to be understood that other examples may be utilized and structural or logical changes may be made without departing from the scope of the present disclosure. The following detailed description, therefore, is not to be taken in a limiting sense. It is to be understood that features of the various examples described herein may be combined, in part or whole, with each other, unless specifically noted otherwise.

At least some examples of the present disclosure are directed to systems and devices for diagnosis, therapy and/or other care of medical conditions. At least some examples may comprise implantable medical devices (IMDs) and/or methods comprising use of IMDs.

IMDs may be formed from a variety of different types of materials. In some examples of the present disclosure, at least portions of the IMDs are formed from a thermoset material, such as silicone. A thermoset material may exhibit particular chemical and/or mechanical properties. For example, thermoset materials may be stable, strong, durable, flexible, biocompatible, and/or temperature resistant. Further, thermoset materials may be formed using a variety of different techniques, including but not limited to, extruding, injection molding, compression molding, and dip coating. Such chemical and mechanical properties may render the thermoset materials suitable for different medical and/or implant implementations.

At least some examples of the present disclosure are directed to methods, devices, and/or systems comprising forming at least one lead portion of a medical lead body in a non-linear configuration from a thermoset material. The medical lead body may comprise a generally flexible elongate conduit that carries electrically conductive wires. In some examples, the at least one lead portion may be formed substantially entirely from the thermoset material (e.g., 90%, 95%, 99%) and/or entirely from the thermoset material. As further described herein, the at least one lead portion in the non-linear configuration changes effective lengths in response to a load (e.g., tension, torsion, compression, shear forces) provided on the medical lead body, which may provide patient comfort when moving.

At least some examples of present disclosure are directed to methods, devices and/or systems comprising forming a body supporting at least one contact electrode. In some examples, the body at least partially defines a re-closable lumen and comprises engaging portions. In some examples, at least one of the engaging portions is formed from a thermoset material, such as a silicone material. In various examples, one of the engaging portions, each of the engaging portions and/or the entire body may be formed from the silicone material. In some examples of the present disclosure, the engaging portions of the body may intersect one another (e.g., are in contact) to at least partially define the re-closable lumen.

In the absence of examples of the present disclosure, when two independent engaging portions formed from silicone material are in contact, a chemical reaction may occur that results in irreversible binding of the silicone material caused by cross-linking of polymers of the silicone material, sometimes herein referred to as “silicone blocking”. In some instances, binding between silicone material of the engaging portions may effectively fuse the engaging portions together, such that the engaging portions may not be separated or there is difficulty separating for implantation and/or sizing purposes of the device.

However, via at least some examples of the present disclosure, forming the body may comprise forming a surface-contact feature on a surface of at least one of the engaging portions. The surface-contact feature may impact the surface contact area between the engaging portions, and may mitigate or prevent irreversible or non-desired binding between silicone material of the engaging portions. For example, the surface-contact feature may be a sufficient size to prevent the engaging portions from binding or fusing together when in contact with one another, such as during transportation, packaging, storage and/or implantation of the body in a patient, while allowing for contact between the engaging portions while the body is being and/or is implanted in the patient.

In some examples, the devices, systems, and methods of the present disclosure are configured and used for sleep disordered breathing (SDB) care, such as obstructive sleep apnea (OSA) care, which may comprise monitoring, diagnosis, and/or stimulation therapy. However, in other examples, the system is used for other types of care and/or therapy, comprising, but not limited to, other types of neurostimulation or cardiac care or therapy. In some examples, such other implementations comprise care, such as but not limited to, central sleep apnea, complex sleep apnea, cardiac disorders, pain management, seizures, deep brain stimulation, respiratory disorders, urinary and/or pelvic disorders.

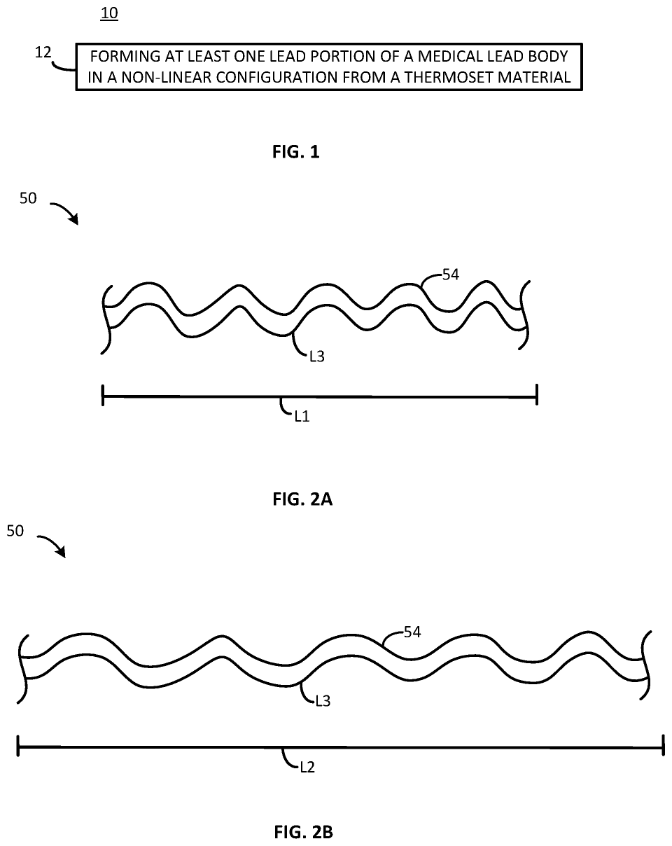

These examples, and additional examples, are described in association with at least FIGS. 1-26C.

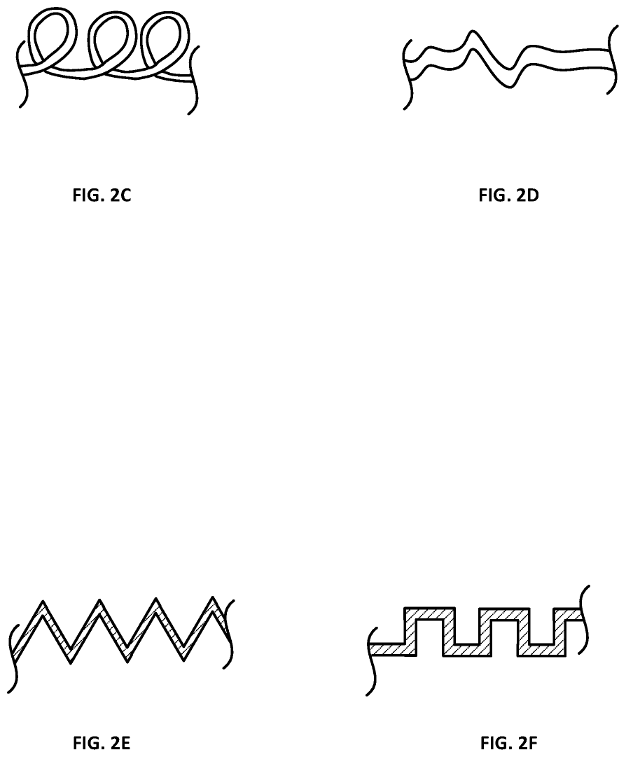

FIG. 1 is a flow diagram schematically representing an example method 10 comprising forming at least a lead portion of a medical lead body in a non-linear configuration. The method 10 comprises forming at least one lead portion of the medical lead body in a non-linear configuration from a thermoset material, as shown at 12 in FIG. 1. In some examples, the non-linear configuration may comprise a generally serpentine configuration, however, examples are not so limited. For example, as further illustrated by FIGS. 2A-2F, the non-linear configuration may comprise a generally sigmoid (or sinusoidal) pattern, a generally helix pattern, a non-continuous sigmoid pattern, a triangular step function pattern, and square wave pattern, among other types of non-linear shapes.

The medical lead body, as used herein, includes or refers to a generally flexible elongate conduit that carries electrically conductive wires. As further described herein, in some examples, the medical lead body may extend from an implantable pulse generator (IPG) at a first end to an operative element at a second end. In some examples, the operative element may comprise a contact electrode, such as a sensing electrode or a stimulating electrode. However, it will be understood that in some examples, the operative element may perform sensing and/or stimulation without being in contact with a particular bodily tissue (e.g. nerve, muscle, etc.) being sensed and/or stimulated.

In some example, the at least one lead portion, in accordance with method 10, may be formed substantially entirely from the thermoset material. Substantially entirely, in various examples, comprises 90 percent (%) of the thermoset material. In some examples, substantially entirely comprises 95% of the thermoset material and, in further examples, comprises 99% of the thermoset material. In various examples, the at least one lead portion is formed entirely from the thermoset material.

In some examples, the method 10 may comprise forming the at least one lead portion from the thermoset material and without a thermoplastic co-layer, such as a polyurethane co-layer. In various examples, the method 10 comprises forming the at least one lead portion from the thermoset material and with a thermoplastic co-layer. As used herein, a thermoset material includes and/or refers to a material that is permanently and/or irreversibly formed (e.g., hardened) from a soft solid or viscous liquid to a hardened shape, such as hardening or curing a polymer from a soft solid or viscous liquid prepolymer or resin. Thermoset materials may contain polymers that cross-link together during the curing process to form an irreversible chemical bond. Example thermoset materials include silicone and silicone blends, such as silicone-polyurethane blends. Thermoplastic materials, as used herein, includes and/or refers to a material that is non-permanently and/or reversibly formed from a soft or viscous liquid to a hardened shape. With thermoplastic materials, the curing process is reversible and the thermoplastic materials may be heated, remolded and/or recycled. Example thermoplastic materials include an elastomer, polyurethane, polyether ether ketone (PEEK), a silicone-polyurethane blend, ethylene tetrafluoroethylene (ETFE), polysulfone, and various combinations thereof.

Various IMDs may comprise a lead including a medical lead body positioned at a location within a human body that may be difficult to position and/or is associated with patient movement. An example is an IMD for SDB, such as for sleep apnea. Sleep apnea generally refers to the cessation of breathing during sleep. One type of sleep apnea, referred to as obstructive sleep apnea (OSA), is characterized by repetitive pauses in breathing during sleep due to the obstruction and/or collapse of the upper airway, and is usually accompanied by a reduction in blood oxygenation saturation.

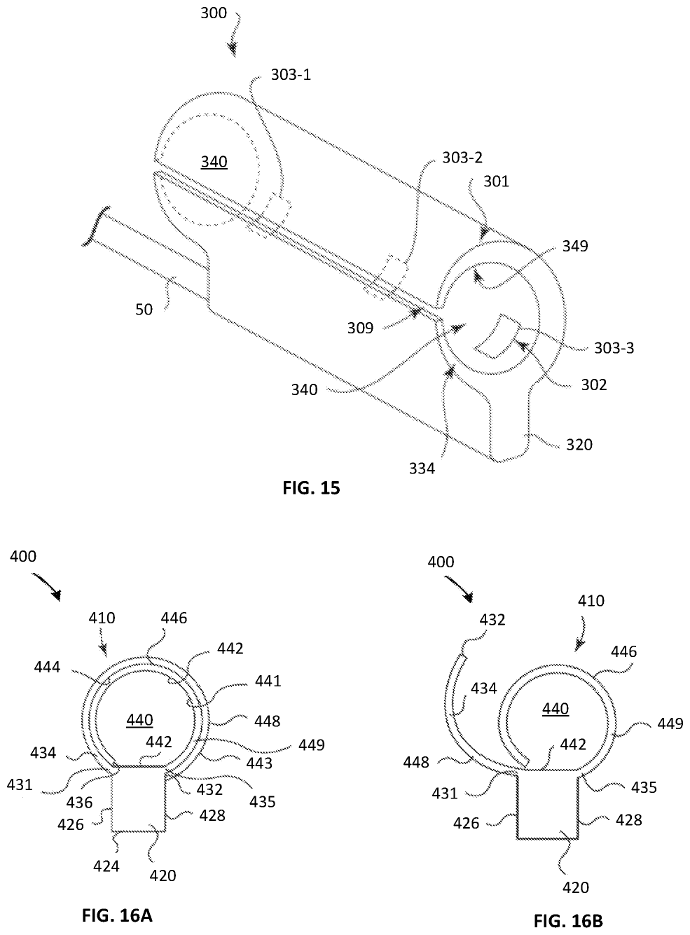

Example treatment for OSA includes the delivery of electrical stimulation to the hypoglossal nerve, located in the neck region under the chin. Such stimulation therapy activates the upper airway muscles to maintain upper airway patency. In treatment of sleep apnea, increased respiratory effort resulting from the difficulty in breathing through an obstructed airway is avoided by stimulation of an upper airway muscle or muscle group that holds the airway open during at least a portion of the inspiratory phase of breathing. For example, the genioglossus muscle is stimulated during treatment of sleep apnea by an electrode cuff placed around the hypoglossal nerve, such as electrode cuff 300 further illustrated herein by at least FIG. 15. In some examples, the stimulation may be synchronized relative to the inspiratory phase of breathing.

Because of the significant amount of movement in multiple directions that may take place under the chin during normal daily living of patient, positioning an electrode to enable stimulation of the hypoglossal nerve may become a challenge. On the one hand, placement of the electrode and lead in close proximity to the hypoglossal nerve may result in irritation to the nerve as a result of normal motion of the chin and neck. On the other hand, without close adherence of the electrode to the nerve, buildup of connective tissue between the nerve and the electrode cuff and/or lead may occur, causing a reduction in the amplitude and thereby the effectiveness of the delivered stimulation by the device. Similarly, a related challenge in maintaining the proper positioning of the electrode includes making a proper placement of a medical lead body that extends from the electrode to the IPG, which may be located in the pectoral region of the patient. Such challenges are not limited to obstructive sleep apnea devices and may apply to other types of care providing IMDs.

The non-linear configuration of the at least one lead portion of the medical lead body formed from the thermoset material may allow for greater patient comfort and ease in lead placement due the flexibility in effective length of the at least one lead portion. For example, a lead portion in a non-linear configuration may change effective lengths (e.g., end-to-end lengths) in response to a load provided on the lead body, which may be caused by patient movement. Thermoset materials may allow for shaping the at least one lead portion in the non-linear configuration, and for providing patient comfort, while keeping bio-stability of the lead. For example, the flexibility in effective length of the at least one lead portion may mitigate or prevent strain exerted on the nerve of the patient in response to the load provided on the medical lead body. In addition, the thermoset material may be durable and/or bio-stabile to allow for a greater lifespan of the medical lead as compared to other less-durable material. A greater lifespan of an IMD and/or IMD components may decrease the number of replacement procedures for the patient.

As further described herein, the method 10 may comprise a number of additional steps and/or variations.

FIGS. 2A-2F illustrate example devices comprising at least one lead portion of a medical lead body in a non-linear configuration. FIGS. 2A-2B illustrate two effective lengths (e.g., end-to-end lengths) of a medical lead body 50 having at least one lead portion 54 in a non-linear configuration. The lead portion 54, in the examples of FIGS. 2A-2B, is in a generally serpentine configuration. In some examples, the serpentine configuration may comprise a sinusoidal pattern or may comprise a generally sigmoid pattern, which may comprise a series of S-shaped curves. FIG. 2A, for example, illustrates the at least one lead portion 54 comprising a first effective length (L1) when in a relaxed state. FIG. 2B illustrates the at least one lead portion 54 comprising a second effective length (L2) when in a deformed state in response to a load provided on the medical lead body 50 with the deformation acting to prevent or relieve strain (e.g. undesired pulling forces) on a nerve or other tissue to which the lead is connected. As shown in FIGS. 2A and 2B, the second effective length (L2) is longer than the first effective length (L1). In one aspect, the at least one lead portion 54 comprises the same arcuate length (L3) (e.g., the length following the S-shaped curves) in the relaxed state as illustrated by FIG. 2A and when a load (e.g., tension, compression, shear, bending, torsion) is provided on the medical lead body 50 as illustrated by FIG. 2B. The at least one lead portion 54 may return to the first effective length (L1) once the load is removed and/or may change to a different effective length in response to a different magnitude and/or direction of load provided on the medical lead body 50. The at least one lead portion 54 may retain the non-linear configuration, in response to the different loads, to provide strain relief upon patient movement of the patient head.

However, examples are not limited to locating the lead in the neck or near the head of the patient. For example, the non-linear configuration allows for substantial patient movement in the IMD, such as for locations of the neck, the arms, legs, or other locations of the patient.

Additionally, examples are not limited to a serpentine non-linear configuration and the least one lead portion 54 may comprise other undulating or curvaceous shapes and patterns. For example, FIG. 2C illustrates an example lead portion of a medical lead body in a generally helix pattern. FIG. 2D illustrates an example lead portion of a medical lead body in a non-continuous sigmoid pattern. FIG. 2E illustrates an example lead portion in a generally triangular step pattern and FIG. 2F illustrates an example lead portion in a generally square wave pattern.

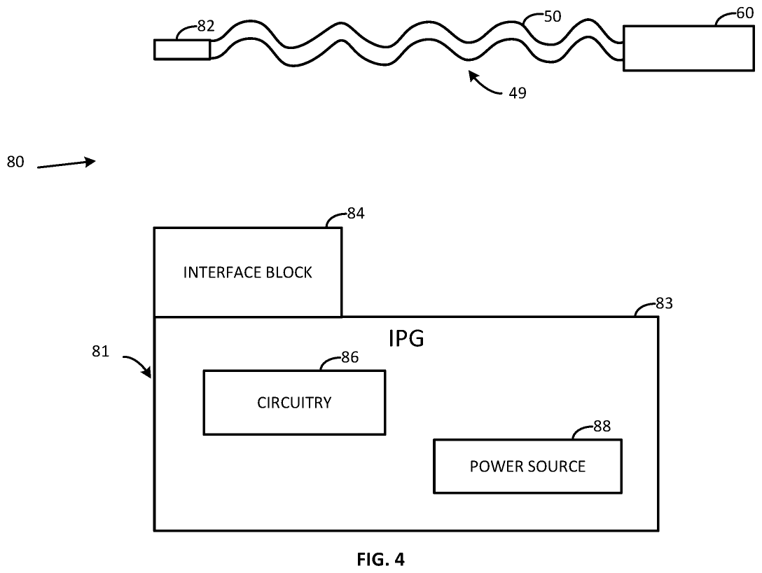

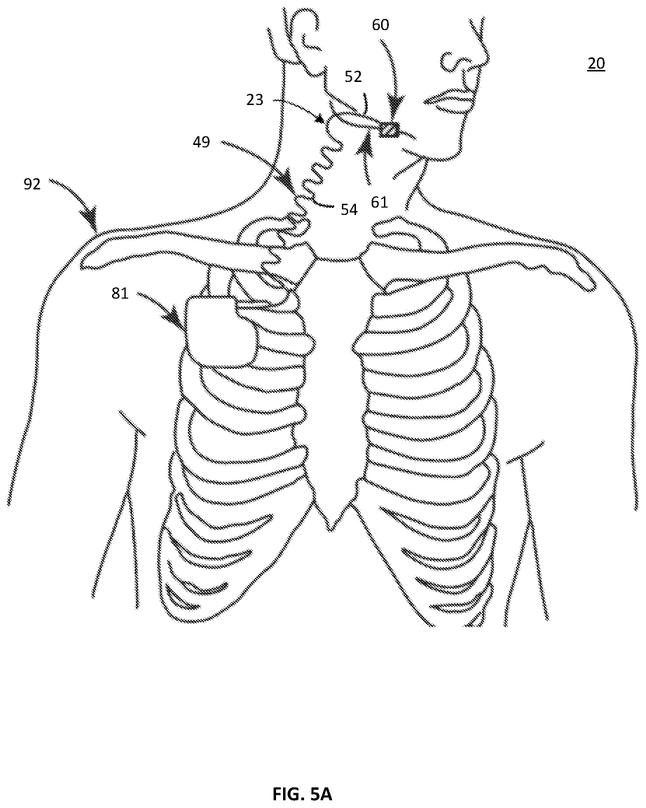

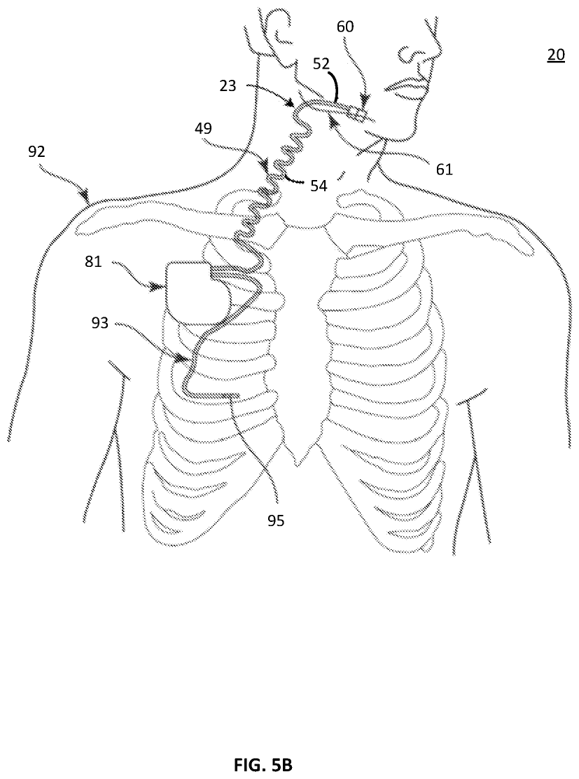

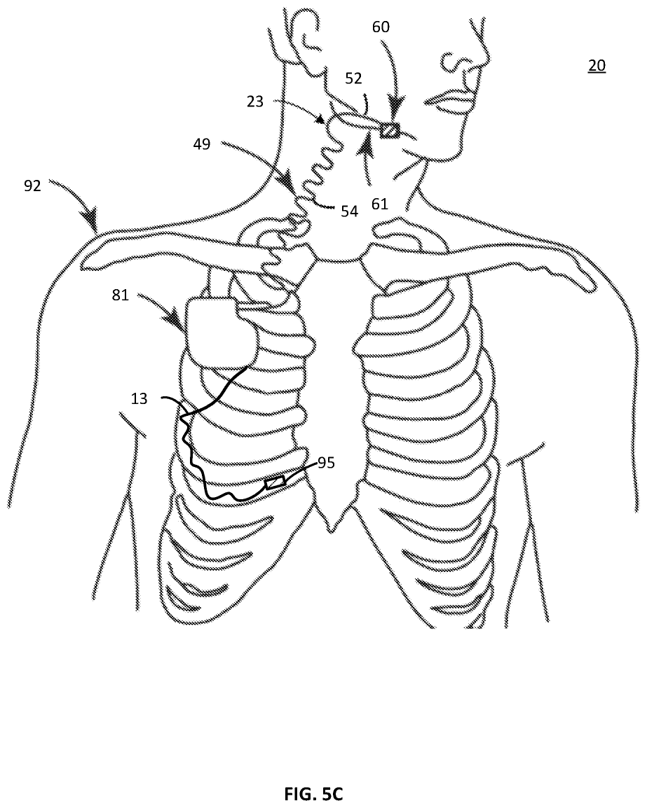

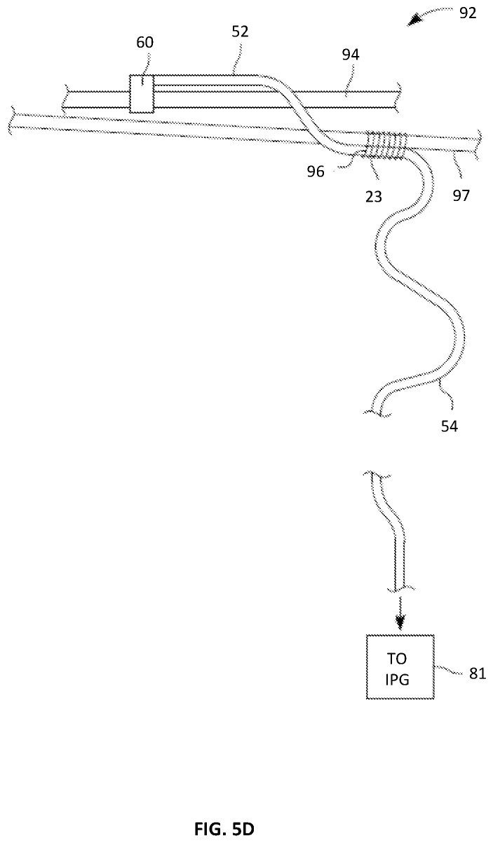

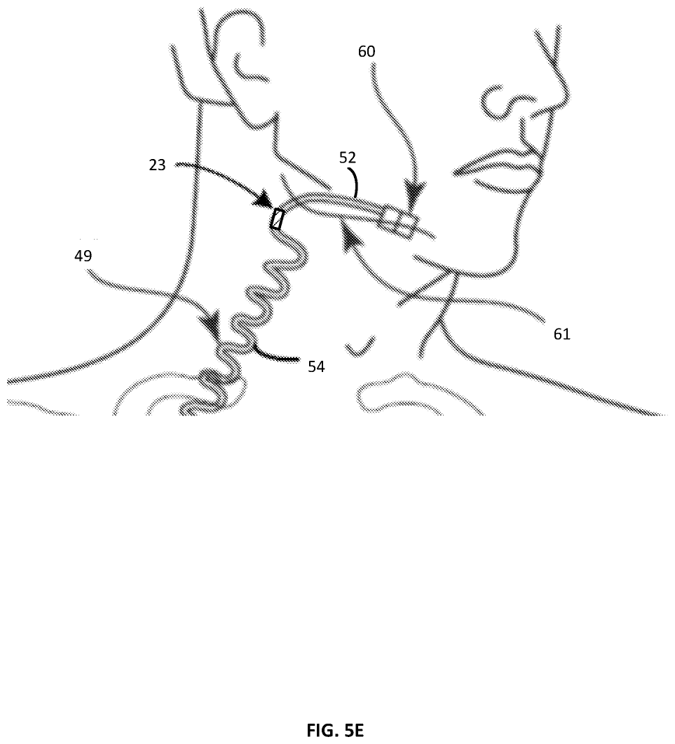

In some examples, the medical lead body 50 of FIGS. 2A-2B may form part of an IMD which may comprise at least some of substantially the same features and attributes as the medical lead body 50 and an IPG 81 as further described in association with at least FIG. 4. The IMD, including the medical lead body 50, may be configured for implantation into a patient, and is configured to provide and/or assist in providing care to the patient, such as illustrated herein by FIGS. 5A-5E.

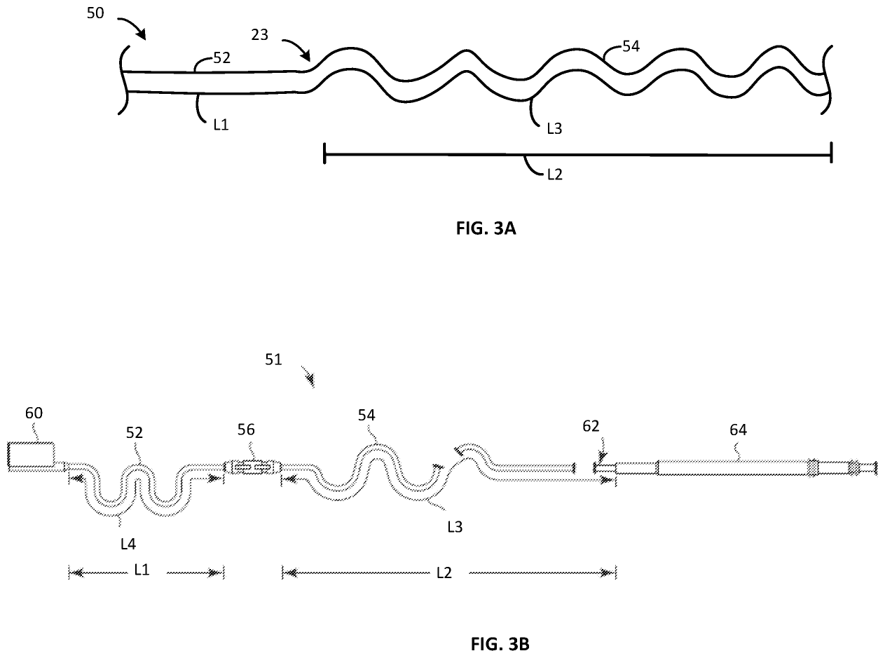

FIGS. 3A-3B illustrate side views of two different example medical lead bodies comprising at least two lead portions 52, 54. As shown by FIG. 3A, in some examples the medical lead body 50 may comprise a first lead portion 52 extending from a second lead portion 54. In some examples, each of the first lead portion 52 and the second lead portion 54 may be formed from the thermoset material. In some examples, the first lead portion 52 and second lead portion 54 are formed as a unitary element. As shown by FIG. 3A, in some examples, the first lead portion 52 may be formed in a linear configuration and the second lead portion 54 may be formed in a non-linear configuration. In some such examples, the medical lead body 50 comprises a transition portion 23 between the first lead portion 52 and the second lead portion 54. In some examples, the transition portion 23 may correspond to a location at which the medical lead body 50 is anchored relative to surrounding non-nerve tissue within a patient's body via an anchoring element and/or other means. In some examples, the lead body 50 may be formed in a manner by which the transition portion 23 comprises or incorporates an anchoring element.

At least some example deployments of medical lead body 50 are shown later in association with at least FIGS. 5A-5E such that an operative element 60 (e.g. cuff electrode, paddle electrode, etc.) may be located at the distal end of the first lead portion 52.

In some examples, the second lead portion 54 may have an effective length (L2) that is substantially longer than the effective length (L1) of the first lead portion 52, however, examples are not so limited. As illustrated, in some examples, the second lead portion 54 may have an arcuate length (L3) that is longer than the effective length (L2) of the second lead portion 54, due to the series of S-shaped curves. The arcuate length corresponds to the length of the second lead portion 54 from end-to-end if the second lead portion 54 were stretched out to eliminate the curved portions. In this arrangement, as further illustrated herein by FIGS. 5A-5C, in some examples the second lead portion 54 has a length sufficient to extend from an anchoring location associated with the transition portion 23 (which may be proximate to the location of an operative element 60 (e.g. at a target nerve 61 as further illustrated by FIGS. 5A-5E), to the location of placement of the IPG 81, such as but not limited to a pectoral region.

However, examples are not so limited, and in some examples the first lead portion 52 may be formed in a non-linear configuration and the second lead portion 54 may be formed in the non-linear configuration from the thermoset material, as illustrated further by FIG. 3B. In some examples, a respective one of the first and second lead portions 52, 54 has a first type of undulating or curvaceous pattern while the other respective one of the first and second lead portions 52, 54 have a second, different type of undulating or curvaceous pattern.

FIG. 3B illustrates a side view of an example lead system 51. In such examples, the lead system 51 comprises an operative element 60 and a medical lead body comprising a first lead portion 52 and a second lead portion 54 formed from a thermoset material. In some examples, both the first lead portion 52 and the second lead portion 54 are in the non-linear configuration. In some examples, the second lead portion 54 may have an effective length (L2) that is substantially longer than the effective length (L1) of the first lead portion 52, however, examples are not so limited. As illustrated, in some examples, the first lead portion 52 may have an arcuate length (L4) that is longer than the effective length (L1) of the first lead portion 52, due to the series of S-shaped curves. In some examples, the operative element 60 comprises a contact electrode. Example contact electrodes include a sensing electrode and a stimulation electrode. In some examples, the operative element 60 comprises an electrode cuff.

In some examples, the lead system 51 further comprises a transition portion 56 between the first lead portion 52 and the second lead portion 54. The transition portion 56 may comprise an anchor configured to be secured relative to a body structure of a patient. In some examples, the body structure may be adjacent to a target nerve on which the operative element 60 is mounted, however, examples are not so limited. In some examples, the lead system 51 may be deployed in a manner which comprises at least some of substantially the same features and attributes as the implantable IMD system 92 as further described in association with at least FIG. 5A, except with the first lead portion 52 of FIG. 3B comprising a non-linear configuration (prior to implantation) which is not depicted in FIGS. 5A-5E. A connector 64 may extend proximally from the second lead portion 54 and is configured to electrically connect to an IPG. In some examples, the lead system 51 further comprises a second transition portion interposed between the second lead portion 54 and the connector 64 at the location represented by reference numeral 62 (or another location closer to the transition portion 56). The second transition portion may comprise a second anchor to be secured related to a second body structure and to provide strain relief for the patient of the lead system 51 relative to the IPG and the first anchor (which is secured in close proximity to the operative element 60).

FIG. 4 is a block diagram schematically representing an example IMD 80. The IMD 80 may comprise an IPG assembly 81 and at least one lead 49. The IPG assembly 81 may comprise a housing 83 containing circuitry 86 and a power source 88 (e.g., battery), and an interface block or header-connector 84 carried or formed by the housing 83. The housing 83 is configured to render the IPG assembly 81 appropriate for implantation into a human body, and may incorporate biocompatible materials and hermetic seal(s). The circuitry 86 may comprise circuitry components and wiring appropriate for generating desired stimulation signals (e.g., converting energy provided by the power source 88 into a desired stimulation signal), for example in the form of a stimulation engine. In some examples, the circuitry 86 may comprise telemetry components for communication with external devices. In some examples, the circuitry 86 may include antennas or features for re-charging a battery.

In some examples, the lead 49 comprises a medical lead body 50 comprising a distally located operative element 60 that comprises a contact electrode. At an opposite end of the medical lead body 50, the lead 49 comprises a proximally located plug-in connector 82 which is configured to be removably connectable to the interface block 84. For example, the interface block 84 may optionally comprise or provide a port sized and shaped to receive the plug-in connector 82.

In some examples, the contact electrode of the operative element 60 may comprise a stimulation electrode and/or a sensing electrode. For example, the contact electrode may be an electrode cuff, and may comprise some non-conductive structures biased to (or otherwise configurable to) releasable secure the electrode about a target nerve. Other formats are also acceptable. In some examples, the operative element 60 may comprise an array of electrodes to deliver a stimulation signal to a target nerve. In various examples, the operative element 60 may comprise at least some of substantially the same features and attributes as described within at least U.S. Pat. No. 8,340,785 issued Dec. 25, 2012 and entitled “SELF EXPANDING ELECTRODE CUFF”, U.S. Pat. No. 9,227,053 issued Jan. 5, 2016 and entitled “SELF EXPANDING ELECTRODE CUFF”, U.S. Patent Publication No. 2020/0230412, published Jul. 23, 2020 and entitled “CUFF ELECTRODE”, and/or U.S. Pat. No. 10,286,206 issued May 14, 2019 and entitled “NERVE CUFF”, the entire teachings of each of which are incorporated herein by reference in their entireties.

As described above, in some examples, the medical lead body 50 is a generally flexible elongate conduit having sufficient resilience to enable advancing and maneuvering the medical lead body 50 subcutaneously to place the operative element 60 (e.g., the contact electrode) at a desired location adjacent a nerve, such as an airway-patency-related nerve (e.g., hypoglossal nerve, phrenic nerve, ansa cervicalis nerve, etc.). In some examples, such as in the case of OSA, the nerves may include (but are not limited to) the nerve and associated muscles responsible for causing movement of the tongue and related musculature to restore airway patency. In some examples, the nerves may include (but are not limited to) the hypoglossal nerve and the muscles may include (but are not limited to) the genioglossus muscle. In some examples, the medical lead body 50 may have a length sufficient to extend from the IPG assembly 81 implanted in one body location (e.g., pectoral) and to the target stimulation location (e.g., head, neck). Upon generation via the circuitry 86, a stimulation signal is selectively transmitted to the interface block 84 for delivery via the lead 49 to such nerves.

FIGS. 5A-5E are diagrams schematically representing deployment of example IMDs, which comprise at least one lead portion of a medical lead body in a non-linear configuration. More particularly, FIG. 5A illustrates an example of an implantable IMD system 92 that comprises an IPG 81 capable of being surgically positioned within a pectoral region of a patient 20, and a lead 49 electrically coupled with the IPG 81 via a connector (not shown) positioned within a connection port of the IPG 81, as previously illustrated by FIG. 4. However, it will be understood that the implantable IMD system 92 may be deployed in other regions of the body. The lead 49 comprises a lead body including at least one lead portion 54 formed from the thermoset material in the non-linear configuration, as previously described. In various examples, the lead 49 comprises a first lead portion 52 in a linear configuration and a second lead portion 54 in the non-linear configuration, and both the first and second lead portions 52, 54 may be formed from the thermoset material.

In some examples, the lead 49 may comprise or is coupled to an operative element 60, such as a contact electrode or electrode system, and extends from the IPG 81 so that the operative element 60 is position around a desired nerve 61 of the patient 20 and to enable stimulation of the nerve 61, as further described below.

In some examples, an anchor may be placed at or near a transition portion 23 between the first lead portion 52 and the second lead portion 54. It will be understood that the exact position of the transition portion 23 and/or anchor within the patient's body may vary depending upon which anatomical structure to which a portion of the lead 49 may be anchored. In some such example arrangements, the first lead portion 52 (in the linear configuration) extends distally from the transition portion 23 (and associated anchor) to the operative element 60. In some such examples, the linear configuration of the first lead portion 52 may sometimes be referred to as a non-sigmoid portion. As further shown in FIG. 5A, in some such examples, the second lead portion 54 (in the non-linear configuration) extends proximally from the transition portion 23 to the IPG 81. It will be further understood that upon implantation, the first lead portion 52 (in its linear configuration) may sometimes exhibit some arcuate bending as the first lead portion 52 is arranged within and among various anatomical features. However, such arcuate bending (e.g. as shown in FIGS. 5A-5E) does not correspond to the first lead portion 52 having a non-linear configuration resulting from formation of the lead 49 prior to implantation.

In some such examples in which the first lead portion 52 is in a linear configuration and extends distally from the transition portion 23 to the operative element 60 coupled relative to target nerve 61, it will be understood that the linear configuration of the first lead portion 52 may be suitably deployed along or near the jaw bone and/or other non-nerve structures in a manner by which little or no strain is placed on the first lead portion 52, the operative element 60, etc. Accordingly, in some examples, the first lead portion 52 may omit a non-linear configuration to simplify lead construction, for situations in which a non-linear configuration may be unnecessary to minimize strain on tissues, and/or for other purposes.

An example implantable stimulation system in which the lead 49 may be utilized, for example, is described in U.S. Pat. No. 6,572,543, issued Jun. 3, 2003 and entitled “SENSOR, METHOD OF SENSOR IMPLANT AND SYSTEM FOR TREATMENT of RESPIRATORY DISORDERS”, the entire teachings of which are incorporated herein by reference in its entirety. In some non-limiting examples, the IMD system 92 may comprise at least some of substantially the same features and attributes as described within at least U.S. Pat. No. 10,932,682, issued on Mar. 2, 2021 and entitled “METHOD AND APPARATUS FOR SENSING RESPIRATORY PRESSURE IN AN IMPLANTABLE STIMULATION SYSTEM”, and/or U.S. Pat. No. 9,227,055 issued Jan. 5, 2016 and entitled “SELF EXPANDING ELECTRODE CUFF”, the entire teachings of which are incorporated herein by reference in their entireties.

In some examples, prior to implantation, the first lead portion 52 may comprise a non-linear configuration and the second lead portion 54 may comprise a linear configuration, with or without an anchor therebetween.

FIG. 5B is diagram including a front view schematically representing deployment of an example IMD system 92 as previously described by FIG. 5A with the addition of a second lead 93. The second lead 93 may be a sensor lead electrically coupled to the IPG 81 and comprising a medical lead body extending from the IPG 81 to a sensing electrode 95. In some examples, the sensing electrode 95 may be in communication with the IPG 81 and may be implanted in the patient 20. The sensing electrode 95 (e.g., a sensor or transducer) may be positioned in the patient 20 for sensing respiratory effort. In some examples, the second lead 93 may comprise a lead body including at least one lead portion in a non-linear or a linear configuration formed from the thermoset material or other material, as previously described for the first lead 49 in FIG. 5A. In various examples, the second lead 93 is formed from a thermoplastic material and the first lead 49 is formed from the thermoset material. In some examples, the second lead 93 is in a linear configuration and formed from a thermoset material or a thermoplastic material, and the first lead 49 comprises the at least one lead portion 54 in the non-linear configuration formed from the thermoset material. In various examples, the second lead 93 is in a non-linear configuration and formed from a thermoset material or a thermoplastic material, and the first lead 49 comprises the at least one lead portion 54 in the non-linear configuration formed from the thermoset material.

In some examples in which the second lead 93 may comprise a non-linear configuration as noted above, and as illustrated by the second lead 13 of the example IMD system 92 illustrated by FIG. 5C, the second lead 93 may comprise at least some of substantially the same features and attributes as the second lead portion 54 (FIGS. 5A-5C) of the first lead 49 when embodied in a non-linear configuration.

FIG. 5D is a side view of an example lead system. As shown by FIG. 5D, in some examples, portions of the IMD system 92 may be secured to different locations of the patient. For example, the operative element 60 may be placed and/or secure about a target nerve, such as a hypoglossal nerve 94, and the first lead portion 52 (in its linear configuration prior to implantation) is maneuvered and arranged to extend proximally from the operative element 60 to a non-nerve bodily structure 97 (such as a digastric tendon, other tendon, or bony structure) that is in close proximity to the target nerve and through the mylohyoid muscle (not shown). In some examples, a first transition portion 23 is secured (e.g., via sutures 96 or other biocompatible fasteners) to the non-nerve bodily structure 97 adjacent the target nerve. In some examples, the second lead portion 54 may be maneuvered and arranged to extend proximally from the first transition portion 23, which includes or is acting as an anchor, toward an IPG 81, using tunneling tools. In some examples, the connector is used to establish electrical communication and mechanical connection to the IPG 81.

In some non-limiting examples, the IMD system 92 and/or lead system may comprise at least some of substantially the same features and attributes as described within at least U.S. Patent. Publication No. 2011/0160827, published on Jun. 30, 2011 and entitled “ELECTRODE LEAD SYSTEM”, the entire teachings of which are incorporated herein by reference in its entirety. For example, although not illustrated, a second anchor may be connected to the second lead portion 54 and interposed between the first transition portion 23 and the connector, such as connector 64 of the lead system of FIG. 3B, wherein the second anchor is configured to be secured relative to a second non-nerve bodily structure.

FIG. 5E is a side view of part of an example lead system. In some examples, the part of the lead system illustrated by FIG. 5E may comprise or form part of the lead systems 92 illustrated by FIG. 5A, FIG. 5B or FIG. 5C. In some examples, the first lead portion 52 is in a linear configuration and the second lead portion 54 is in a non-linear configuration with the transition portion 23 located therebetween. In some examples, portions of the IMD system 92 may be secured to different locations of the patient and may comprise at least some of substantially the same features and attributes as previously described in association with at least FIG. 5D. For example, the operative element 60 may be placed and/or secure about a target nerve 61, and the first lead portion 52 extends proximally from the operative element 60 to at least a non-nerve bodily structure and sometimes may extend proximally beyond the non-nerve bodily structure. In some examples, the first transition portion 23 (see also FIGS. 5A-5D) between the first lead portion 52 and second lead portion 54 is secured to the non-nerve bodily structure. However, it will be understood that portions of the medical lead 49 instead of or in addition to the transition portion 23 may be secured relative to non-nerve bodily structures. In some examples, such securing may be implemented via anchoring elements coupled relative to portions of the lead 49 and/or incorporated into the lead 49, as previously noted. The second lead portion 54 (e.g., having non-linear configuration) may be maneuvered and arranged to extend proximally from the anchor toward an IPG 81.

While the examples of FIGS. 5A-5E illustrate example IMDs and/or IMD systems comprising the IPG implanted within a pectoral region of a patient 20 and the lead 49 extending from the pectoral region to the neck region of the patient and comprising an operative element that is coupled to a nerve, examples are not so limited.

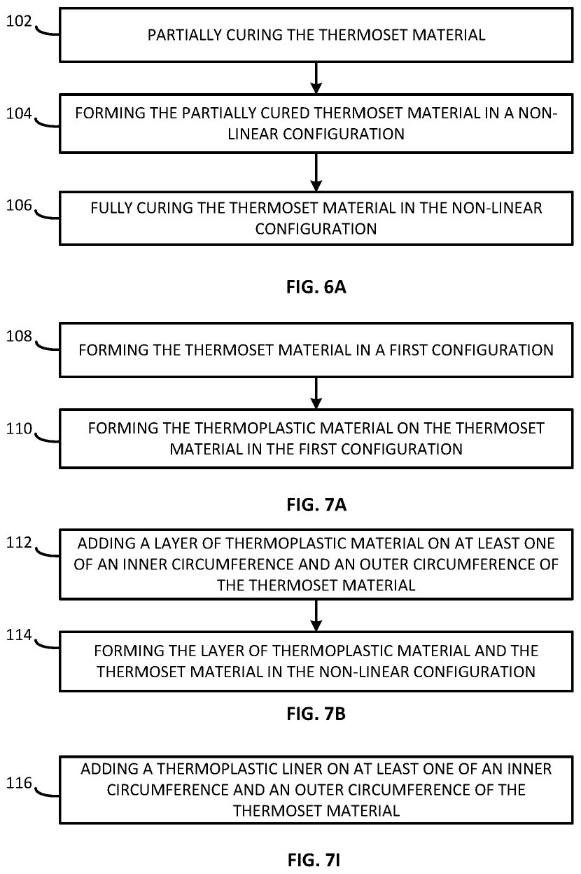

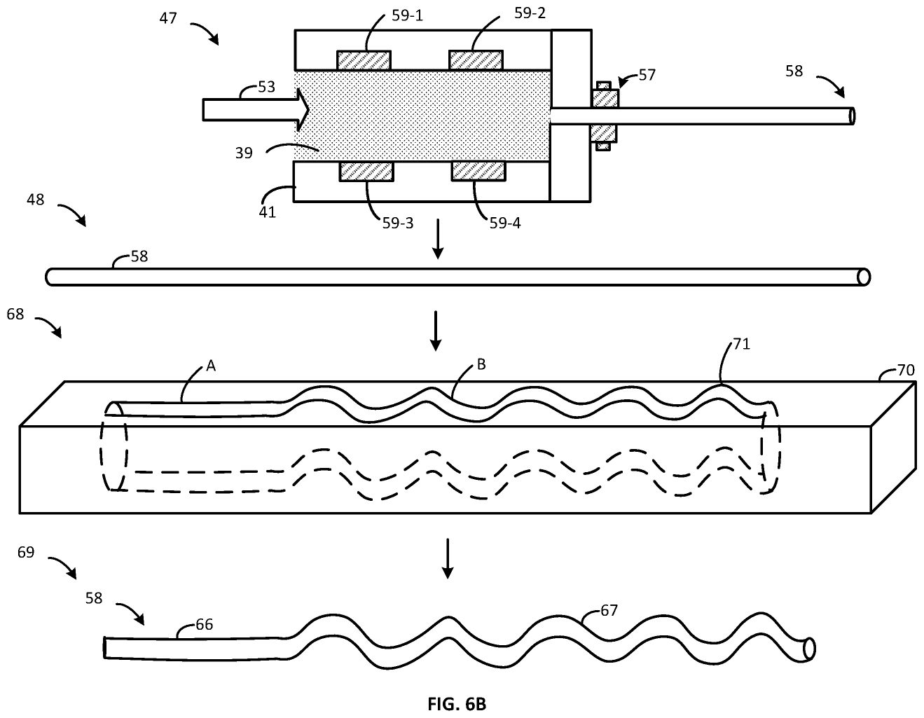

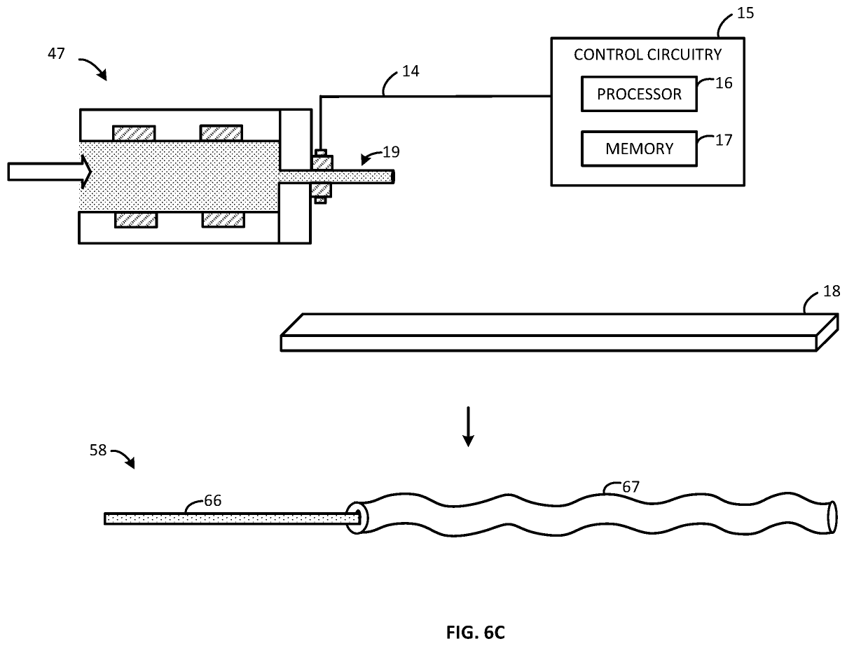

FIGS. 6A-11 illustrate different example methods of forming a medical lead body. In some examples, the methods illustrated by FIGS. 6A-11 may comprise part of and/or are example implementations of the method 10 illustrated by FIG. 1. In some examples, the medical lead bodies illustrated by FIGS. 2A-2F, 3A-3B, 4, and/or 5A-5E may formed by any of the methods described and illustrated by FIGS. 6A-11.

FIGS. 6A-6C are diagrams which may comprise part of and/or are example implementations of a flow diagram in example methods (e.g., method 10). As shown in FIG. 6A, in some examples, forming the at least one lead portion may comprise, at 102 in FIG. 6A, partially curing the thermoset material, at 104, forming the partially cured thermoset material in a non-linear configuration, and at 106, fully curing the thermoset material in the non-linear configuration. Partially curing the thermoset material may comprise extruding the silicone material to a partially cured state. However examples are not so limited and, in some examples, the thermoset material may be molded to the partially cured state. In some examples, the thermoset material may be partially cured by allowing the material to cure for a period of time, with or without the application of heat. The partially cured state may comprise a state between liquid and fully cured, in which the thermoset material may still be manipulated and is not (yet) set, which may sometimes herein referred to as a “green state”. While in the green state, the thermoset material may at least partially hold its shape and may be further manipulated until fully cured or set. In some examples, forming the partially cured thermoset material in the non-linear configuration may comprise molding the partially cured silicone material. For example, the thermoset material in the partially cured form may be placed in a forming tray and the partially cured thermoset material may be allowed to set or fully cure while in the forming tray. The forming tray may comprise a cavity (e.g., negative space) shaped to set the thermoset material in the non-linear configuration, as further illustrated by the forming tray 70 of FIG. 6B.

FIG. 6B illustrates an example implementation of the method of forming the lead body as described and illustrated by FIG. 6A. In some examples, an extruder is used to extrude the thermoset material, as shown at 47 in FIG. 6B. The extruder may comprise a container 41, at least one heater 59-1, 59-2, 59-3, 59-4 (herein generally referred to as “the at least one heater 59” for ease of reference), a die 57, and a force component 53. In some examples, the container 41 holds the extruding material 39, which in the example implementation of FIG. 6B may comprise the thermoset material. The at least one heater 59 heats the extruding material 39 to a particular temperature sufficient to melt the extruding material 39. In some examples, the die 57 comprises a cross-sectional profile (e.g., shaped cutouts and/or cavities) that shapes the heated extruding material 39 to an intended configuration, and the force component 53 may force the heated extruding material 39 toward and through the die 57. In some examples, the force component 53 may comprise a ram on an opposite side of the container 41 as the die 57. The ram may move toward the die 57 during extrusion to force the extruding material 39 through the die 57 to form a conduit 58 from the extruding material 39. In some examples, the force component 53 may comprise a rotating screw that is located across the container 41 and that rotates to move the extruding material 39 toward and through the die 57 to form the conduit 58. In some examples, the at least one heater 59 is located on the rotating screw.

In some examples, the conduit 58 formed from thermoset material is allowed to partially cured in the linear configuration, as shown at 48 in FIG. 6B. As shown at 68 in FIG. 6B, in some examples, the conduit 58, which is partially cured, may be placed in a forming tray 70 that comprises a cavity 71 shaped in the non-linear configuration for the at least one lead portion of the conduit 58. In some examples, the cavity 71 comprises a first sub-portion shaped in a linear configuration, as illustrated by A in FIG. 6B, and a second sub-portion shaped in the non-linear configuration, as illustrated by B in FIG. 6B. While in the forming tray 70, the thermoset material may be fully cured or set in the non-linear configuration (and optionally, the linear configuration) to form the lead body. In some examples, as shown at 69 in FIG. 6B, the resulting conduit 58 is formed entirely of thermoset material, and comprises a first lead portion 66 in a linear configuration and a second lead portion 67 in a non-linear configuration. However, examples are not so limited and, in some examples, the entire conduit 58 may be in the non-linear configuration.

In some examples, as shown by FIG. 6C, forming the partially cured thermoset material in the non-linear configuration comprises moving an extruding head 19 (e.g., the die 57 or other component near and/or including the die 57 as illustrated by FIG. 6B) of the extruder relative to a forming tray 18 and/or moving the forming tray 18 relative to the extruding head 19 to form the partially cured thermoset material of the at least one lead portion in the non-linear configuration. The extruder and/or forming tray 18 illustrated by FIG. 6C may include at least some of substantially the same attributes and features as the extruder and/or forming tray 70, and the associated extruding of thermoset material, as previously described by FIG. 6B. In some examples, the forming tray 18 may be flat and/or may not include a cavity shaped based on the non-linear configuration. In some examples, the extruder is used to extrude the thermoset material at 47 of FIG. 6C, and as previously described by FIG. 6B, and control circuitry 15 coupled to the extruder and/or forming tray 18 may move the extruding head 19 and the forming tray 18 relative to each other to form the partially cured thermoset material in the non-linear configuration, as shown by the conduit 58 of FIG. 6C which includes a first lead portion 66 in a linear configuration and a second lead portion 67 in a non-linear configuration; however, examples are not so limited as described above. Alternatively, the extruding head 19 or forming tray 18 may be manually moved or otherwise manipulated by a user, and without the use of the control circuitry 15, to form the partially cured thermoset material in the non-linear configuration. For example, the extruded thermoset material (e.g., conduit 58) may exit the extruder in a linear configuration, and the at least one lead portion of the lead body may be in the linear configuration for a (brief) period of time and allowed to partially cure before motion of the extruding head (e.g., die 57 of FIG. 6B) relative to the forming tray 18 causes the at least one lead portion to be in the non-linear configuration, such as shown by 69 of FIG. 6C. While in non-linear configuration, the thermoset material may be cured to the fully cured state. However, examples are not so limited, and in some examples, forming the partially cured thermoset material in the non-linear configuration comprises manually manipulating the partially cured thermoset, which may be extruded or molded to the partially cured state.

In some examples, the control circuitry 15 includes a processor 16 and memory 17 that stores machine readable instructions. In some examples, the control circuitry 15 may be in communication with the extruder, such as via a wired or wireless communication link 14. However examples are not so limited, and the control circuitry 15 may be in communication with a mechanical component that is attached to the extruding head 19 and/or the forming tray 18. The processor 16 may execute the machine readable instructions stored on the memory 17, which when executed cause the processor 16 to perform the above-identified actions, such as causing the extruding head 19 and/or the forming tray 18 to move relative to each other to form the partially cured thermoset material in the non-linear configuration.

In some examples, the machine readable instructions may be loaded in a random access memory (RAM) for execution by the processor 16 from their stored location in a read only memory (ROM), a mass storage device, or some other persistent storage (e.g., non-transitory tangible medium or non-volatile tangible medium), as represented by memory 17. Example memories include ROM, RAM, flash memory, a solid state drive, and/or discrete data register sets. In some examples, the machine readable instructions may comprise a sequence of instructions, a processor-executable model, or the like. In some examples, the memory 17 may be a non-transitory storage medium, where the term “non-transitory” does not encompass transitory propagating signals. In some examples, the memory 17 comprises a machine readable tangible medium providing non-volatile storage of the machine readable instructions executable by the processor 16 of the control circuitry 15. In some examples, the machine readable tangible medium may sometimes be referred to as, and/or comprise at least a portion of, a computer program product. In some examples, hard wired circuitry may be used in place of or in combination with machine readable instructions to implement the functions described. In some examples, the control circuitry 15 may be embodied as part of at least one application-specific integrated circuit (ASIC), at least one field-programmable gate array (FPGA), and/or the like. In at least some examples, the control circuitry 15 is not limited to any specific combination of hardware circuitry and machine readable instructions, nor limited to any particular source for the machine readable instructions executed by the processor 16. In some examples, control circuitry 15 may be entirely implemented within or by a stand-alone device.

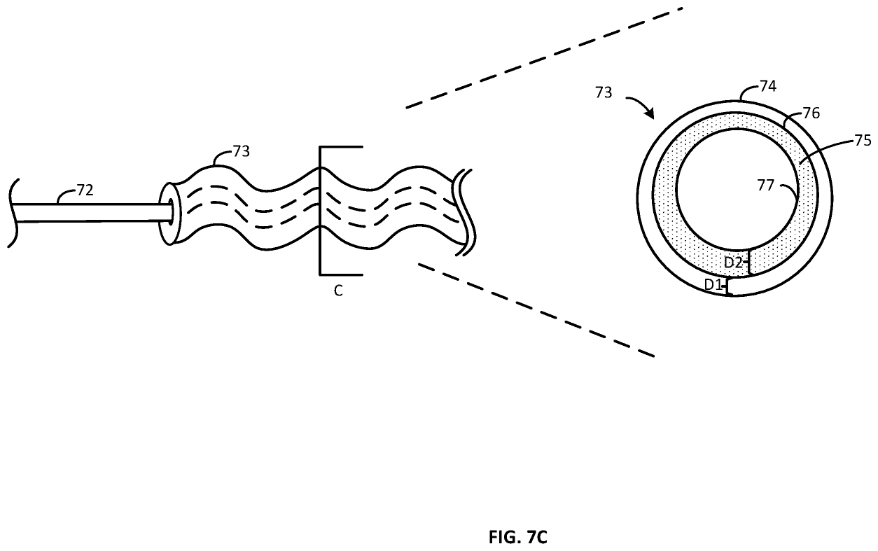

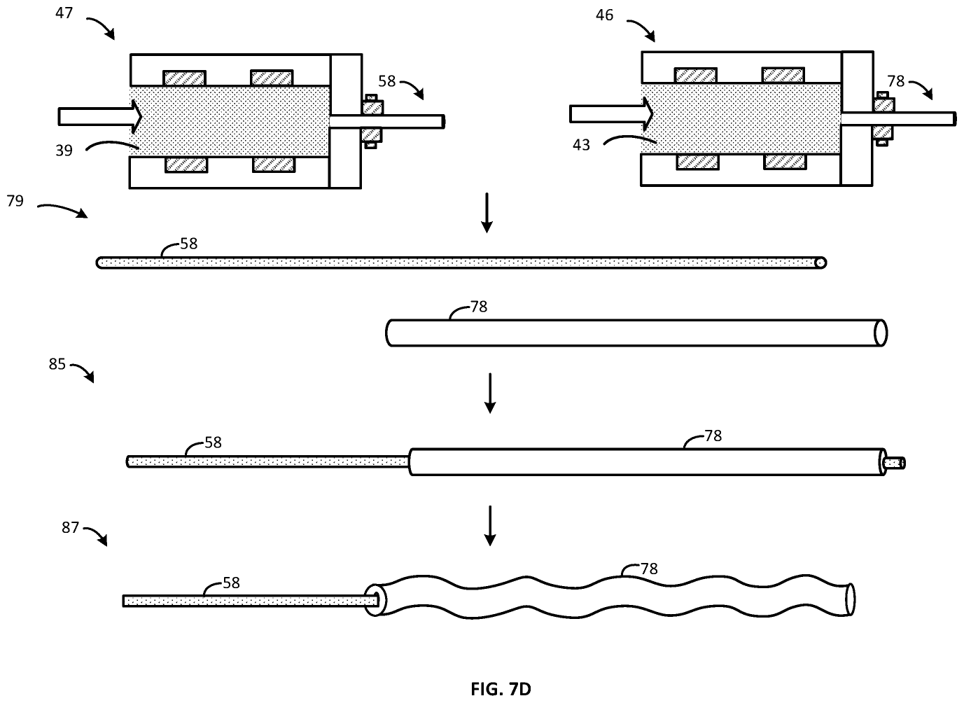

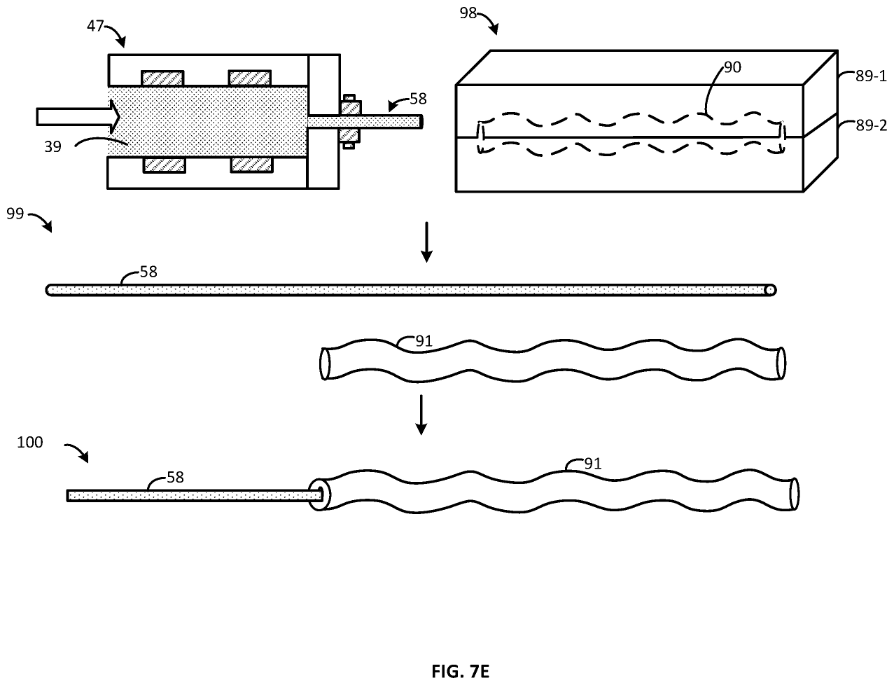

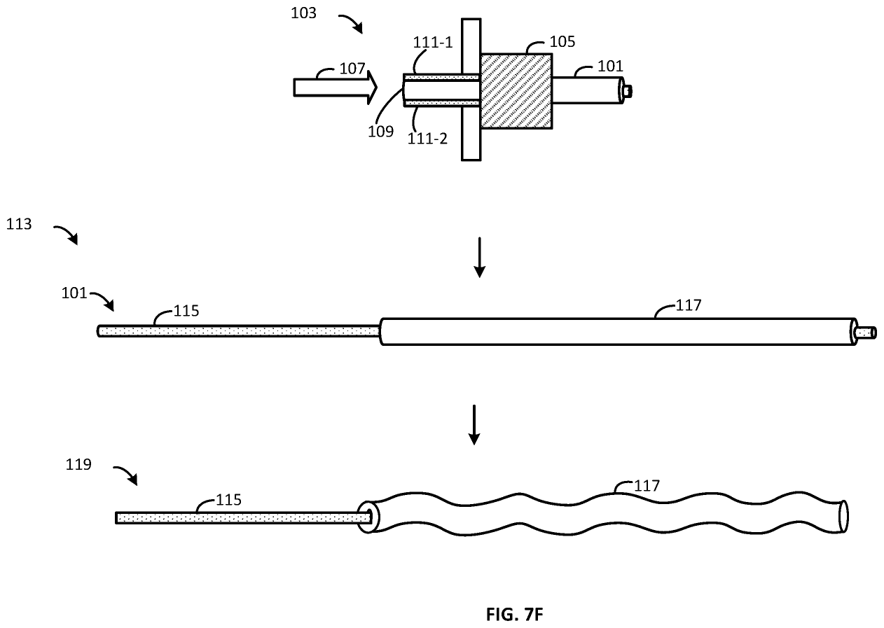

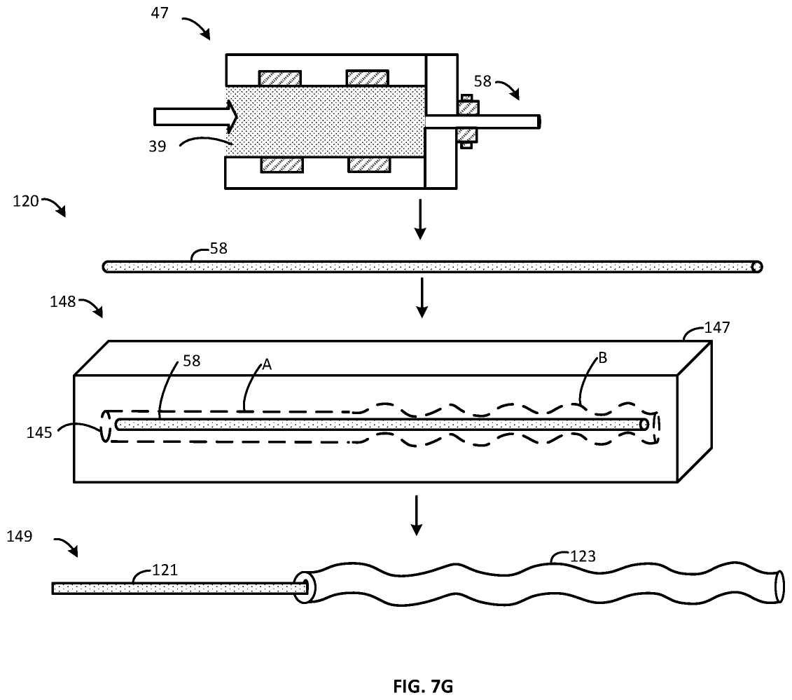

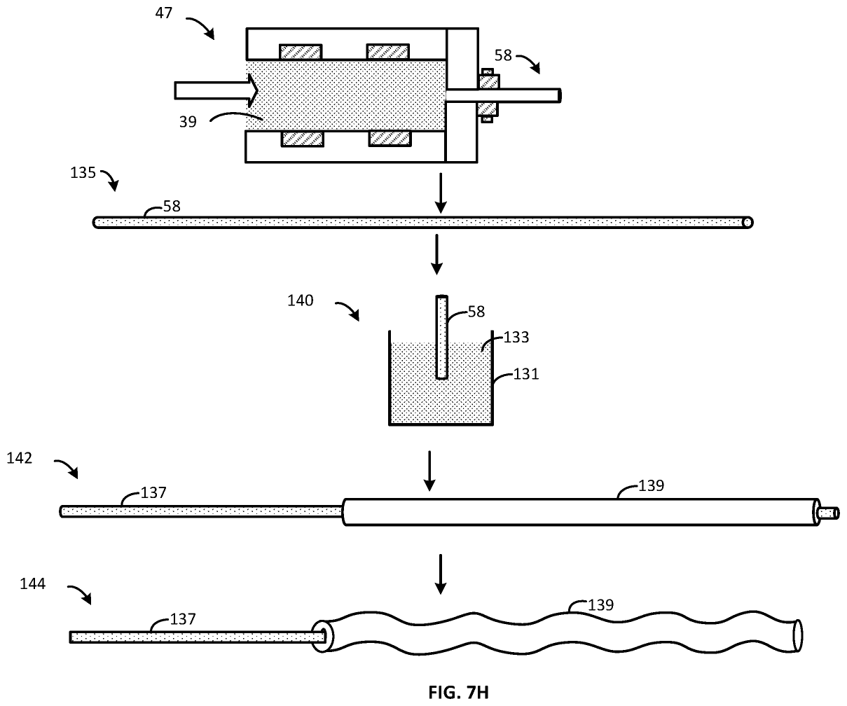

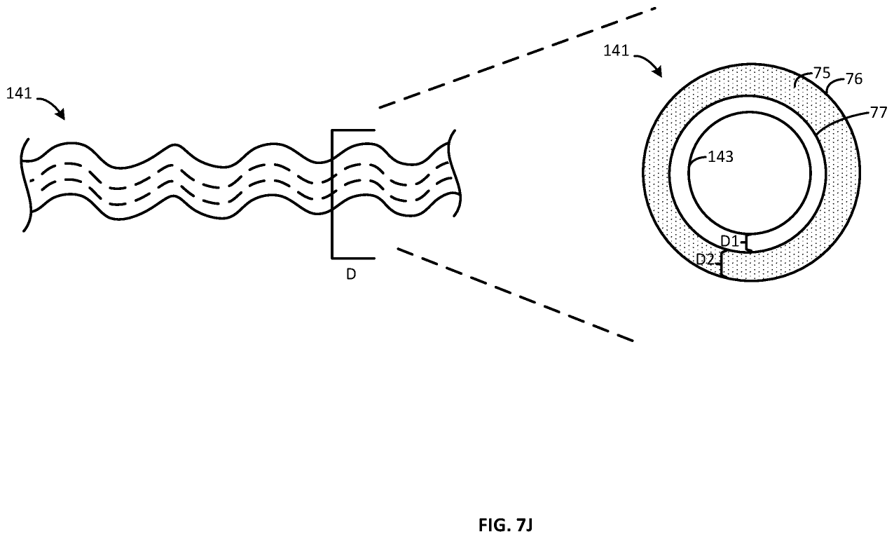

FIGS. 7A-7J are diagrams, which may comprise part of and/or are example implementations of a flow diagram in an example method (e.g., method 10). As shown in FIG. 7A, forming the at least one lead portion may comprise, at 108, forming the thermoset material in a first configuration, and at 110, forming a thermoplastic material on the thermoset material in the first configuration to form the at least one lead portion in the non-linear configuration. The first configuration, in various examples, is a linear configuration (e.g., a straight conduit) and forming the thermoset material may comprise extruding or molding the thermoset material as a conduit in the linear configuration. In some examples, the thermoplastic material may form a liner or jacket on at least one of the outer circumference or the inner circumference of the thermoset material in the first configuration. In some examples, the thermoplastic material is formed as a solid rod, tube, or other shaped element.

In some examples, forming the thermoplastic material on the thermoset material in the method of FIG. 7A, as illustrated in FIG. 7B, may comprise, at 112, adding a layer of thermoplastic material on at least one of an inner circumference (e.g., 77 of FIG. 7C) and an outer circumference (e.g., 76 of FIG. 7C) of the thermoset material in the linear configuration for the at least one lead portion. And, at 114, the method further comprises forming the layer thermoplastic material and the thermoset material in the non-linear configuration for the at least one lead portion. In various examples, the at least one lead portion is a sub-portion of the overall geometry of the medical lead body, as shown by FIG. 7C, and in some examples, is the entire geometry of the medical lead body, as shown by FIG. 7J. In some examples, the thermoplastic material may be shaped to fit on the inner circumference (e.g., smaller than the inner circumference 77 as shown by FIG. 7J) or the outer circumference (e.g., larger than the outer circumference 76 as shown by FIG. 7C) of the at least one lead portion formed from the thermoset material. In such examples, the thermoplastic material may be a jacket or a liner for the at least one lead portion.

FIG. 7C illustrates an example of a lead body formed according to an example implementation of the method of FIG. 7B. As shown in FIG. 7C, the lead body may comprise a first lead portion 72 in a linear configuration (and without a thermoset co-layer) and a second lead portion 73 in a non-linear configuration with a thermoplastic liner. FIG. 7C additionally illustrates a cross-sectional view of the second lead portion 73, taken along line C. In some examples, the second lead portion 73 comprises a thermoplastic liner 74 located on the outer circumference 76 of the thermoset material 75 (e.g., a conduit formed from the thermoset material).

In some examples of the method illustrated by FIG. 7B, adding the layer of thermoplastic material may comprise forming the thermoplastic material for the at least one lead portion in a linear configuration and assembling the thermoplastic material formed in the linear configuration as the layer on the thermoset material in the linear configuration, such as further illustrated by FIG. 7D. In such examples, forming the layer of the thermoplastic material and the thermoset material in the non-linear configuration may comprise shaping the assembled thermoplastic material and thermoset material in the non-linear configuration for the at least one lead portion, such as by using heat and/or a mold.

FIG. 7D illustrates an example method of forming a lead body by assembling the thermoplastic material as a layer on thermoset material and shaping the thermoplastic material, which in some examples may comprise an example implementation of the method described by FIG. 7B. In some examples, two linear conduits 58, 78 are formed by separately extruding the conduits 58, 78 as shown at 47 and 46 in FIG. 7D. In some examples, extruding the thermoset material 39, at 47 in FIG. 7D, and the thermoplastic material 43, at 46 in FIG. 7D, may comprise at least some of substantially the same attributes and features as extruding the thermoset material as previously described at 47 as associated with at least FIG. 6B, with the extruded material comprising the thermoplastic material 43 for 46 in FIG. 7D. As shown at 79 in FIG. 7D, a first linear conduit 58 (e.g., a straight thermoset tube) may be formed from the thermoset material 39, and a second linear conduit 78 (e.g., a straight thermoplastic tube) may be formed from the thermoplastic material 43 via the extrusion processes. The second linear conduit 78 may comprise a length of the at least one lead portion of the first linear conduit 58, which may be a sub-portion of or the entire length of the first linear conduit 58, in various examples. In some examples, the two linear conduits 58, 78 are assembled together, with the second linear conduit 78 being placed relative to the at least one lead portion of the first linear conduit 58 (e.g., on the outer or inner circumference), as shown at 85 in FIG. 7D. The second linear conduit 78 may then be shaped to the non-linear configuration via the application of heat, as shown at 87 in FIG. 7D, which may cause the at least one lead portion of the assembled first and second linear conduits 58, 78 to exhibit a non-linear configuration. However, examples are not limited to extruding the linear conduits 58, 78. In some examples, one or more of the linear conduits 58, 78 may alternatively be formed from a molding process, such as injection molding and/or compression molding as further illustrated by FIG. 7E.

In further examples of the method illustrated by FIG. 7B, adding the layer of thermoplastic material and forming the layer of the thermoplastic material and the thermoset material in the non-linear configuration comprises forming the thermoplastic material in the non-linear configuration for the at least one lead portion and assembling the thermoplastic material formed in the linear configuration as the layer on the thermoset material in the linear configuration as illustrated by FIG. 7E.

FIG. 7E illustrates an example implementation of a method of forming a lead body by assembling the thermoplastic material as a layer on thermoset material, which in some examples may comprise an example implementation of the method described by FIG. 7B. In some examples, as shown at 47 and 98 in FIG. 7E, two conduits 58, 91 are formed. In some examples, as shown at 47 in FIG. 7E, the first linear conduit 58 (e.g., a straight thermoset tube) may be formed by extruding the thermoset material 39, which in some examples may comprise at least some of substantially the same attributes and features as extruding the thermoset material 39 as previously described at 47 in association with at least FIG. 6B. In some examples, the second non-linear conduit 91 (e.g., sigmoid thermoplastic tube) may be formed from the thermoplastic material via a molding process, as shown at 98 in FIG. 7E. In some example, the thermoplastic material may be compression molded or injection molded. For compression molding, at least one mold 89-1, 89-2 may be used to heat the molding material (e.g., the thermoplastic) at appropriate locations and/or to apply force to the molding material to shape the material into the intended configuration. The at least one mold 89-1, 89-2 may comprise a cavity 90 shaped to set the configuration (e.g., a negative form of the configuration). For injection molding, the molding material is heated and pressure forced into a cavity 90 of the mold 89-1, 89-2 through an orifice in the mold. However examples are not limited to the second non-linear conduit 91 being formed of a thermoplastic material, and in some examples, a thermoset material is molded to form the second non-linear conduit 91.

In either molding process, the thermoplastic material may be cured while in the mold 89-1, 89-2 by cooling down the material. As shown in 99 of FIG. 7E, in some examples, the second non-linear conduit 91 has a length of the at least one lead portion of the first linear conduit 58. In some examples, as shown at 100 in FIG. 7E, the two conduits 58, 91 may be assembled together, with the second non-linear conduit 91 being placed relative to the at least one lead portion of the first linear conduit 58 (e.g., on the outer or inner circumference). In some examples, the resulting non-linear configuration of the least one lead portion may be an intermediate configuration that is between the linear configuration of the thermoset material (e.g., conduit 58 as shown at 99 in FIG. 7E) and the non-linear configuration of the thermoplastic material (e.g., conduit 91 as shown at 99 in FIG. 7E).

In various examples of the method illustrated by FIG. 7B, forming the thermoset material in the linear configuration and adding the layer of thermoplastic material may comprise concurrently extruding a first layer of the thermoset material and a second layer of the thermoplastic layer in the linear configuration. In such examples, the first layer of thermoset material may comprises a first conduit and the second layer of thermoplastic material comprises a second conduit formed as a liner on one of the inner circumference and the outer circumference of the first conduit. In such examples, forming the layer of the thermoplastic material and the thermoset material in the non-linear configuration may comprise shaping the first and second layers of thermoplastic material and thermoset material in the non-linear configuration for the at least one lead portion, such as by using heat and/or a mold.

FIG. 7F illustrates an example implementation of a method of forming a lead body by co-extruding the thermoplastic material and the thermoset material, which in some examples may comprise an example implementation of the method described by FIG. 7B. For example, as shown at 103 in FIG. 7F, the first and second layers of thermoplastic material and thermoset material may be co-extruded, resulting in a conduit 101 in the linear configuration. In some examples, the conduit 101 comprises at least one lead portion comprising the co-layers of the thermoset material 109 and the thermoplastic material 111-1, 111-2. In some examples, the co-extrusion process may comprise at least some of substantially the same features or attributes of the extrusion process 47 as previously described in association with at least FIG. 6B, but with the extruder comprising at least two containers to contain the thermoset material 109 and the thermoplastic material 111-1 and 111-2, and the die 105 being shaped to extrude the conduit 101 from the thermoset material 109 with a co-layer of thermoplastic material 111-1, 111-2. A force component 107 may force the thermoset material 109 and the thermoplastic material 111-1, 111-2 through the die 105. In some examples, although not illustrated, the force component 107 may comprise a first force component that forces the thermoset material 109 through the die 105 and a second (or more) force component that forces the thermoplastic material 111-1, 111-2 the die 105. In some examples, the first and second (or more) force components may be separately controlled.

In some examples, as shown at 113 in FIG. 7F, the co-extruded conduit 101 may comprise a first lead portion 115 formed from the thermoset material 109 and a second lead portion 117 formed from the thermoset material 109 and the thermoplastic material 111-1, 111-2 (e.g., a straight tube having two layers of material). In some examples, as shown at 119 in FIG. 7F, at least one lead portion of the conduit 101 may be shaped into the non-linear configuration. In some examples, the at least one portion comprises a sub-portion of the entire lead body, such as the second lead portion 117 illustrated at 119 in FIG. 7F, and the first lead portion 115 may be in a linear configuration and may not include a layer of the thermoplastic material.

In some examples of the method illustrated by FIG. 7B, adding the layer of thermoplastic material and forming the layer of the thermoplastic material and the thermoset material in the non-linear configuration comprises molding the layer of thermoplastic material in the non-linear configuration on at least one of the inner circumference and the outer circumference of the thermoset material in the linear configuration as shown by FIG. 7G.

FIG. 7G illustrates an example implementation of a method of forming a lead body by molding the thermoplastic material on the thermoset material, which in some examples may comprise an example implementation of the method described by FIG. 7B. As shown at 47 and at 120 in FIG. 7G, a linear conduit 58 (e.g., a straight thermoset tube) may be formed from the thermoset material via an extrusion process. In some examples, the extrusion process at 47 may comprise at least some of substantially the same features and attributes as the extrusion procession 47 previously described in association with at least FIG. 6B.

In some examples, as shown at 148 in FIG. 7G, the linear conduit 58 may be placed in a mold 147 or a tray. The mold 147 may comprise a cavity 145 shaped to set the thermoplastic material in the non-linear configuration (e.g., a negative form of the configuration) for the at least one lead portion. In some examples, the cavity 145 of the mold 147 may be shaped such that the linear conduit is not in contact with the top or bottom of the cavity 145 and the thermoplastic material may form around the outer circumference of the linear conduit 58. In some examples, the thermoplastic material is placed in the cavity 145 and heated for compression molding, or is heated and injected into the cavity 145 via an orifice for injection molding, as shown at 148 in FIG. 7G. In some examples, the cavity 145 comprises a first sub-portion shaped in a linear configuration, as illustrated by A in FIG. 7G, and a second sub-portion shaped in the non-linear configuration, as illustrated by B in FIG. 7G.

However, examples are not limited to molding a thermoplastic material, and various examples may include molding a thermoset material and/or a thermoplastic material into a non-linear configuration. In some examples, the at least one lead portion is formed from a thermoset material that is molded to form the non-linear configuration, and the at least one lead portion may not include a layer of thermoplastic material. In some examples, the at least one lead portion is formed from a thermoset material extruded in a linear configuration and then assembled with a co-layer of thermoset material molded into the non-linear configuration.

In some examples, the molding process illustrated by FIG. 7G may add a layer of thermoplastic material around one of the inner circumference (e.g., 77 in FIG. 7C) and the outer circumference (e.g., 76 in FIG. 7C) of the conduit 58 formed from the thermoset material. In some examples, the layer of thermoplastic material may be cured to a non-linear configuration around the linear conduit for the at least one lead portion of the conduit 58, and as shown at 149 in FIG. 7G, resulting in the at least one lead portion of the conduit 58 being shaped in the non-linear configuration (e.g., a non-straight tube having two layers of material). In some examples, the at least one lead portion comprises a sub-portion of the entire lead body, such as the second lead portion 123 illustrated at 149, and the first lead portion 121 may be in a linear configuration and may not include a layer of the thermoplastic material.

In some examples of the method illustrated by FIG. 7B, adding the layer of thermoplastic material may comprise dip coating the thermoplastic material as the layer on the thermoset material in the linear configuration. In such examples, forming the layer of the thermoplastic material and the thermoset material in the non-linear configuration may comprise shaping the thermoplastic material and thermoset material in the non-linear configuration for the at least one lead portion, such as by using heat.

FIG. 7H illustrates an example implementation of a method of forming a lead body by dip coating the thermoplastic material on the thermoset material, which in some examples may comprise an example implementation of the method described by FIG. 7B. In some examples, as shown at 47 and at 135 in FIG. 7H, a linear conduit 58 (e.g., a straight thermoset tube) may be formed from the thermoset material 39 via an extrusion process, which in some examples may comprise at least some of substantially the same features and attributes as the extrusion process 47 previously described in association with at least FIG. 6B. In some examples, as shown at 140 in FIG. 7H, the linear conduit 58 may be immersed, at least partially, into a container 131 that contains a solution 133 comprising the thermoplastic material. In some examples, the thermoplastic material coats on a surface (e.g., outer circumference and/or inner circumference) of the linear conduit 58, and then the linear conduit 58 is removed from the solution 133, resulting in a layer of thermoset material on a surface of at least one lead portion of the linear conduit 58.

As shown at 142 in FIG. 7H, in some examples, a first lead portion 137 is formed from the thermoset material (and without a co-layer of thermoplastic material) and a second lead portion 139 is formed from the thermoset material and comprises a co-layer of thermoplastic material. The at least one lead portion comprising the thermoset material and thermoplastic material (e.g., the second lead portion 139) may be shaped into the non-linear configuration, as shown at 144 in FIG. 7H. In some examples, shaping the at least one lead portion at 144 in FIG. 7H may comprise at least some of substantially the same features and attributes as previously described in association with at least 119 in FIG. 7F. In some examples, the at least one lead portion comprises a sub-portion of the entire lead body, such as the second lead portion 139 illustrated at 144, and the first lead portion 137 may be in a linear configuration and may not include a layer of the thermoplastic material. However, examples are not so limited, and the thermoset material may be dip coated onto the thermoplastic material in various examples. In some examples, the thermoplastic material may be formed in the non-linear configuration for the at least one lead portion and dip coated in a solution comprising the thermoset material.

In various examples of the method illustrated by FIG. 7B, as illustrated by FIG. 7I, adding the layer of the thermoplastic material on the thermoset material may comprise, as shown at 116 in FIG. 7I, adding a thermoplastic liner to at least one of an inner circumference (e.g., 77 of FIGS. 7J and/or 7C) and an outer circumference (e.g., 76 of FIGS. 7J and/or 7C) of the thermoset material. In some examples, as illustrated by FIGS. 7C and 7J, the thermoplastic liner 74, 143 may comprise a first thickness D1 and the thermoset material 75 in the first configuration may comprise a second thickness D2, with the first thickness D1 being less than the second thickness D2. The thermoset material 75 in the first configuration and the thermoplastic liner 74 may be formed in a variety of ways, such as those described above in association with at least FIGS. 7A-7B.

While FIGS. 7C, 7E, and 7G illustrate the co-layer of thermoplastic being on the outer circumference of the thermoset material and extending a sub-portion of the length of the lead body, examples are not so limited. In some examples, as illustrated by the cross-sectional view of the lead body 141 (e.g., the conduit) taken along line D in FIG. 7J, the co-layer or liner 143 formed from thermoplastic is on the inner circumference 77 of the thermoset material 75. In some examples, which may be in addition to or alternatively to the thermoplastic co-layer or liner 143 being on the inner circumference 77 of the thermoset material, the thermoplastic co-layer or liner 143 extends the entire length or substantially the entire length of the lead body 141. In some examples, the lead body 141 illustrated by FIG. 7J may be formed according to at least some of substantially the same features and attributes as the methods as previously described in association with at least FIGS. 7A, 7B, and/or 71.

However, examples are not limited to forming a layer of thermoplastic material on the thermoset material and in various examples, the thermoset material is formed in the non-linear configuration by extruding and/or molding the thermoset material, with or without the thermoplastic material. In some examples, the thermoset material is extruded in the non-linear configuration and assembled with a thermoplastic material in the non-linear configuration. In some examples, forming the at least one lead portion comprises concurrently extruding a first layer of the thermoset material and a second layer of the thermoplastic material in the non-linear configuration. In such examples, the thermoplastic material and thermoset material may be co-layers or the thermoplastic material in the non-linear configuration may be a shaped element, as further described herein.

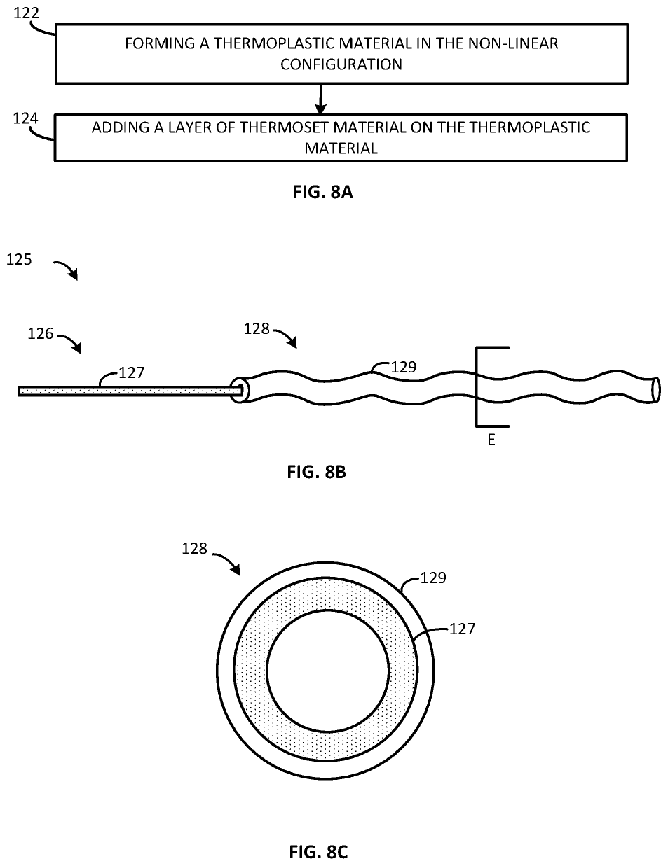

FIGS. 8A-8C are diagrams which may comprise part and/or are example implementations of a flow diagram in an example method, such as forming part of method 10 of FIG. 1. As shown in FIG. 8A, forming the at least one lead portion may comprise, at 122, forming the thermoplastic material in the non-linear configuration for the at least one lead portion, and at 124, adding a layer of the thermoset material on the thermoplastic material in the non-linear configuration to form the at least one lead portion in the non-linear configuration. In some examples, the thermoplastic material in the non-linear configuration comprises a shaped element, such as the shaped element 129 as shown by FIG. 8B.

FIGS. 8B-8C illustrates an example implementation of a method of FIG. 8A. As shown in FIG. 8B, the shaped element 129 may form part of the at least one lead portion 128 of the medical lead body 125. In some examples, the medical lead body 125 comprises a first lead portion 126 formed of the thermoset material 127 in a linear configuration, and the second lead portion 128 formed of the thermoset material 127 and the shaped element 129 in the non-linear configuration. In some examples, the method of FIG. 8A may further comprise removing the shaped element 129. FIG. 8C illustrates a cross-sectional view of the at least one lead portion 128 along the line E of FIG. 8B and which illustrates the shaped element 129 on the outer circumference of thermoset material 127.

Adding the layer of thermoset material, in accordance with the method of FIG. 8A, may comprise extruding the thermoset material in a linear configuration and assembling the thermoset material with the thermoplastic material, which in some examples may comprise at least some of substantially the same features and attributes as the method previously described in association with at least FIG. 7D. In other examples, adding the layer of thermoset comprises molding the layer of the thermoset material on at least one of an outer circumference and an inner circumference of the thermoplastic material in the non-linear configuration, which in some examples may comprise at least some of substantially the same features and attributes as the method previously described in association with at least FIG. 7G, but with the thermoplastic material formed as a non-linear conduit and the thermoset material molded around a surface of the non-linear conduit. In some examples, adding the layer of thermoset comprises coating (e.g., dip coating) and curing the layer of the thermoset material on at least one of an outer circumference and an inner circumference of the thermoplastic material in the non-linear configuration, which in some examples may comprise at least some of substantially the same features and attributes as the method previously described in association with at least FIG. 7H.

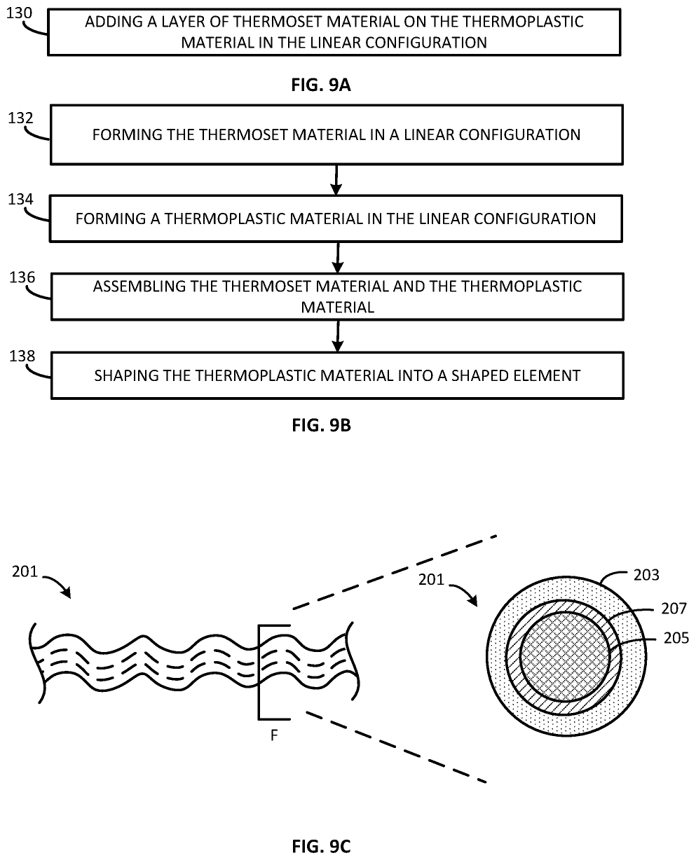

FIGS. 9A-9C are diagrams which may comprise part of and/or are example implementations of a flow diagram in an example method (e.g., method 10). As shown at 130 in FIG. 9A, forming the at least one lead portion may comprise adding a layer of the thermoset material on a thermoplastic material in a linear configuration to form the at least one lead portion in the non-linear configuration. In some examples, the thermoplastic material in the linear configuration comprises a solid shaped element, and the method further comprises shaping the thermoplastic material into the non-linear configuration and/or removing the shaped element. In such examples, the shaped element comprises a stylet. In some examples, the shaped element forms part of the at least one lead portion of the medical lead body, such as a thermoplastic rod.

As noted above, the method may further comprise shaping the thermoplastic material into the non-linear configuration to form the shaped element. For example, as shown at 132 in FIG. 9B, forming the at least one lead portion may comprise forming the thermoset material in a linear configuration for the at least one lead portion, and at 134, forming a thermoplastic material in the linear configuration. In some examples, the method may further comprise, at 136, assembling the thermoplastic material formed in the linear configuration with the thermoset material formed in the linear configuration for the at least one lead portion, and at 138, shaping the thermoplastic material into a shaped element comprising a first configuration that is non-linear. In some examples, the shaped element of FIG. 9B may comprise at least some of substantially the same features and attributes as the shaped element 129 previously described in association with at least FIG. 8B. As previously described and illustrated by FIGS. 6B, 7D and 7E, the thermoset material and/or the thermoplastic material may be formed in the linear configuration by extruding and/or molding the thermoset and/or thermoplastic materials.

The first configuration, in some examples, comprises the non-linear configuration of the at least one lead portion, however examples are not so limited. In various examples, the assembled thermoplastic material and the thermoset material for the at least one lead portion transforms to an intermediate configuration that is between the first configuration of the shaped element and the linear configuration of the thermoset material. In some examples, the intermediate configuration comprises the non-linear configuration of the at least one lead portion.

FIG. 9C illustrates an example of a lead body comprising at least one lead portion 201 in a non-linear configuration that includes a thermoplastic rod 205 and a thermoset material 203, which in some examples may comprise an example implementation of the method described by FIG. 9A. FIG. 9C further illustrates a cross-sectional view of the at least one lead portion 201 along the line F. As shown, the thermoplastic rod 205 may be positioned proximal to an inner circumference of the thermoset material 203. In such examples, intermediate components 207 (e.g., conductors) may be between the thermoset material 203 and the thermoplastic rod 205.

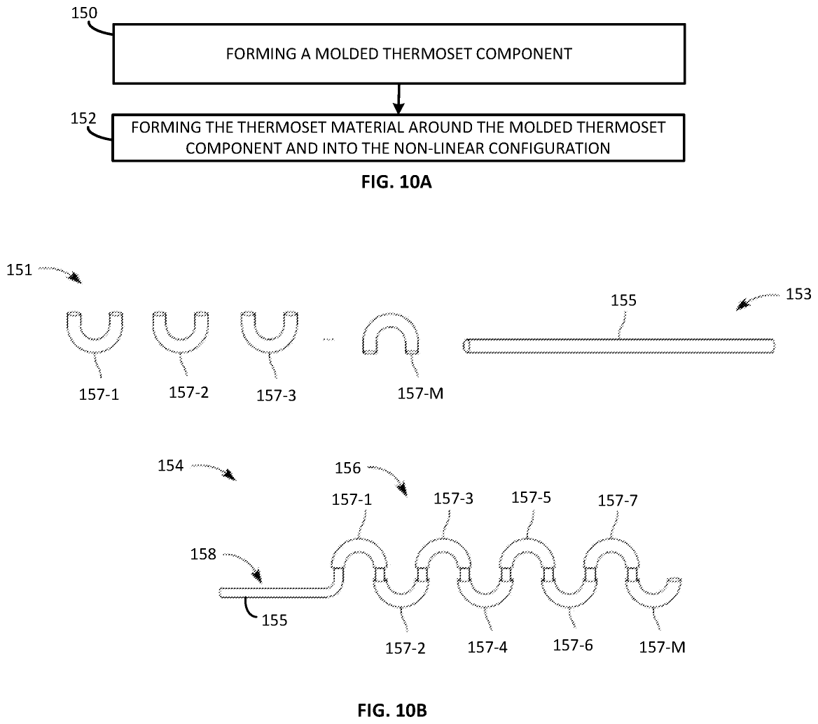

FIGS. 10A-10B are diagrams which may comprise part of and/or are example implementations of a flow diagram in an example method (e.g., method 10). Forming the at least one lead portion, as shown at 150 in FIG. 10A, may comprise forming a molded thermoset component, and at 152, forming the thermoset material around the molded thermoset component and into the non-linear configuration. In some examples, a plurality of molded thermoset components (e.g., short molded components) are formed, which are used as shaped elements to provide the non-linear shape to the thermoset material for the at least one lead portion. Each of the plurality of molded thermoset components may have a length that is significantly shorter than the length of the at least one lead portion of the medical lead body.

FIG. 10B illustrates an example of a plurality of molded thermoset components 157-1, 157-2, 157-3, 157-4 . . . 157-M for forming the at least one lead portion of a lead body in a non-linear configuration, which in some examples may comprise an example implementation of the method described by FIG. 10A. In some examples, such as shown at 153 in FIG. 10B, the lead body 155 is formed from a thermoset material in a linear configuration for the entire length of the lead body. In some examples, the lead body 155 may be formed via an extrusion process and/or molding process which may comprise at least substantially the same features and attributes as previously described in association with at least FIGS. 6B and 7E, with the molded material including a thermoset material. As shown at 151 in FIG. 10B, a plurality of molded thermoset components 157-1, 157-2, 157-3, 157-4 . . . 157-M (herein generally referred to as “the molded thermoset components 157” for ease of reference) may be formed by a molding processing, as previously described in association with at least FIG. 7E, with the molded material including a thermoset material. As shown at 154 in FIG. 10B, the molded thermoset components 157 may be assembled with the lead body 155 to set the at least one portion of the lead body in the non-linear configuration. In some examples, the lead body 155 may comprise a first lead portion 158 in a linear configuration (and without any molded thermoset components) and a second lead portion 156 assembled with the molded thermoset components 157 in the non-linear configuration.

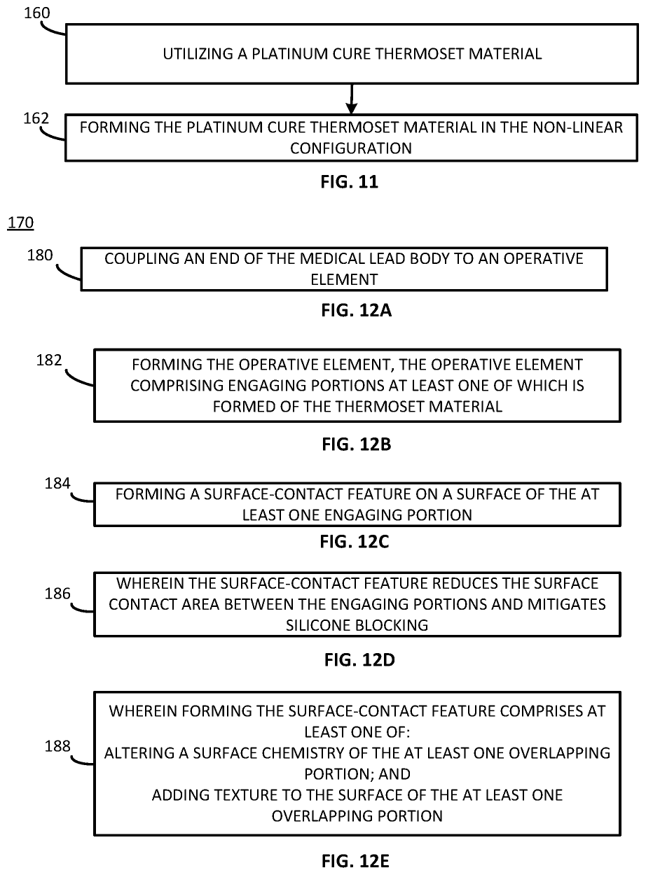

FIG. 11 is a flow diagram which may comprise part of and/or is an example implementation of a flow diagram in an example method (e.g., method 10). In some examples, forming the at least one lead portion, as shown at 160 in FIG. 11, may comprise utilizing a platinum cure thermoset material, and as shown at 162, forming the platinum cured thermoset material in the non-linear configuration.

FIGS. 12A-12E are diagrams, which may comprise part of and/or are example implementations of a flow diagram in an example method 170. In some examples, the method 170 of FIG. 12A may comprise part of method 10 of FIG. 1. In various examples, the lead system may be coupled to an operative element. As shown at 180 in FIG. 12A, the method 170 may comprise coupling an end of the medical lead body to an operative element that comprises at least one contact electrode. In some examples, the lead body may comprise at least some of substantially the same features and attributes as the lead body as previously described in association with at least FIG. 4. In some examples, as shown at 182 in FIG. 12B, the method 170 may further comprise forming the operative element, with the operative element comprising engaging portions and at least one of the engaging portions being formed from a thermoset material, such as a silicone material.

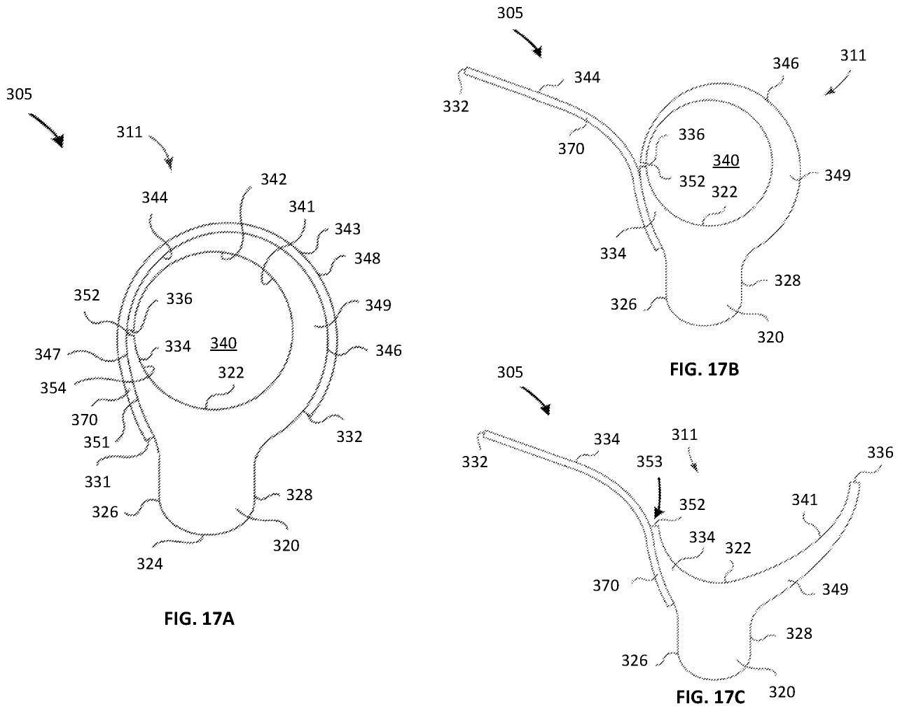

In some examples, the operative element comprises an expandable electrode cuff, however, the examples are not so limited and the operative element may comprise a sensing electrode or other types of electrodes. For example, as shown at 184 in FIG. 12C, forming the electrode cuff may comprise forming at least one arm, the at least one arm comprising a proximal portion that extends from a base of a cuff body outwardly and a distal portion, the engaging portions comprising at least a surface of the at least one arm. In some examples, the electrode cuff may comprise at least some of substantially the same features and attributes as the electrode cuffs 300, 305, 400 as further described in association with at least FIG. 15, FIGS. 16A-16B and/or FIGS. 17A-17C. As further illustrated herein, in some examples, the surface is at least one of an inner surface of the at least one arm, an outer surface of the at least one arm, an abutting end of the at least one arm, and a side surface of the at least one arm.

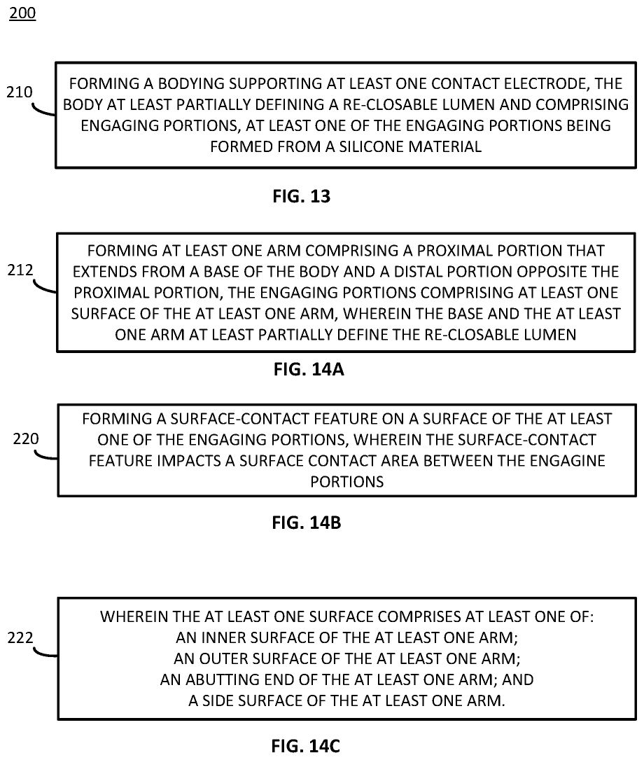

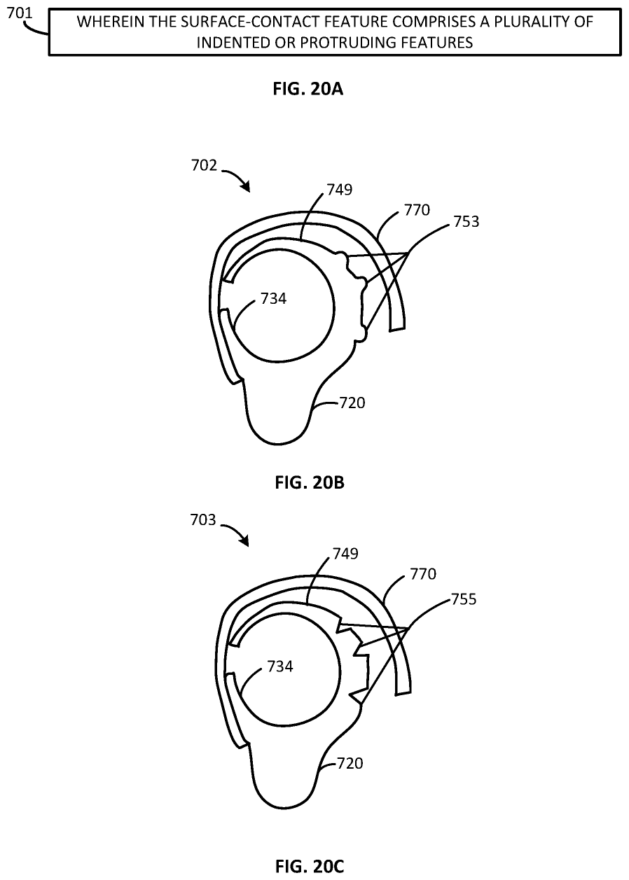

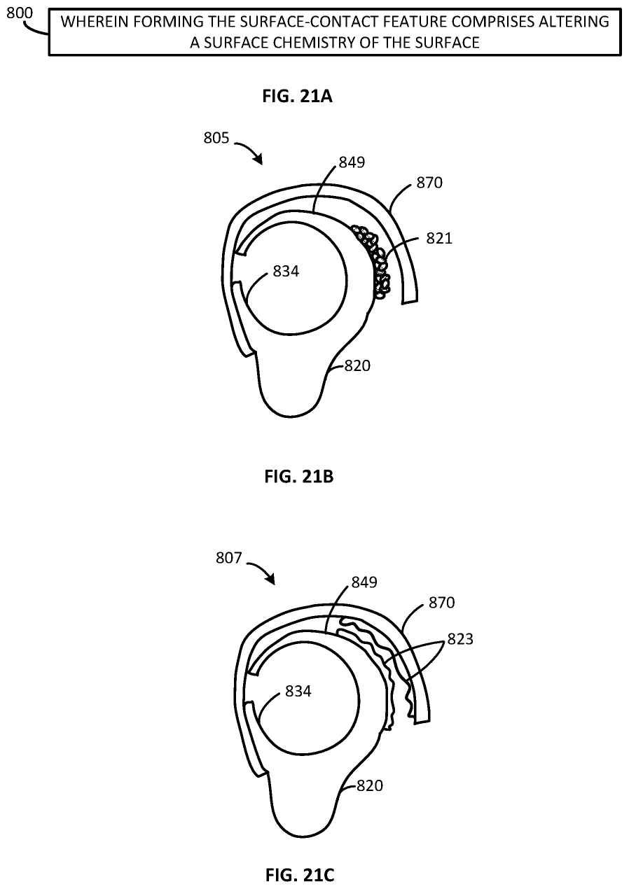

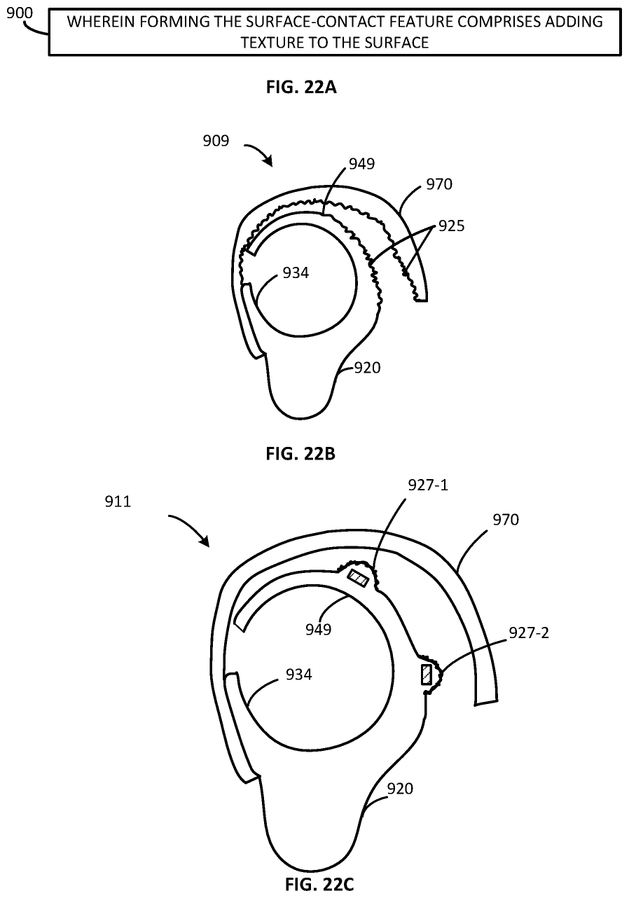

As shown at 186 in FIG. 12D, in some examples, the method 170 may further comprise forming a surface-contact feature on a surface of at least one of the engaging portions. In some examples, the surface-contact feature may comprise at least some of substantially the same features and attributes as the surface-contact features as further described in association with at least FIGS. 20A-20C, FIGS. 21A-21C, and/or FIGS. 22A-22C. In some examples, the surface-contact feature may prevent or mitigate the engaging portions formed from a silicone material from bonding or fusing together when in contact, at least prior to implantation of the device, as further described below in connection with method 200 of FIG. 13. In some examples, the surface-contact feature may impact a surface contact area between the engaging portions. In some examples, the surface-contact feature reduces the surface contact area between the engaging portions and mitigates silicone blocking. In some examples, the surface-contact feature may comprise a plurality of indented or protruding features of a smaller size than a surface area of the engaging portions. In some examples, the protrusions and/or the indentation may comprise at least some of substantially the same features and attributes as the protrusions and/or indentations as further described herein in association with at least at least FIGS. 20B-20C.

In some examples, as shown at 188 in FIG. 12E, forming the surface-contact feature comprises performing at least one of altering a surface chemistry of the surface of the at least one engaging portion, and adding texture to the surface of the at least one engaging portion, as further described herein. In some examples, altering the surface chemistry may comprise at least some of substantially the same features and attributes as the protrusions and/or indentations as further described herein by at least FIGS. 20B-20C, 21B-21C, and/or 22B-22C.

FIG. 13 is a flow diagram schematically representing an example method 200 comprising forming a body supporting at least one contact electrode. In some examples, the method 200 may comprise part of method 10 illustrated by FIG. 1, however, examples are not so limited. As shown at 210 in FIG. 13, the method 200 comprises forming a body supporting at least one contact electrode, the body at least partially defining a re-closable lumen and comprising engaging portions, at least one of the engaging portions being formed from a silicone material. In some examples, the body and at least one electrode comprise an implantable operative element, as described above in connection with FIGS. 12A-12E. In some examples, the body is a cuff body of an electrode cuff.

In some examples, forming the body comprises forming the at least one engaging portion substantially entirely (e.g., such as 90%, 95% or 99%) or entirely from the silicone material. In some examples, each of the engaging portions are formed from the silicone material. In various examples, the body is the cuff body of the electrode cuff and the entire cuff body is formed from the silicone material.

As described above in connection with FIG. 1, components of an IMD may be formed from thermoset material to allow for shaping the component, while keeping bio-stability. At least some thermoset material, such as silicone, may be susceptible to fusing to other silicone material, sometimes herein referred to as “silicone blocking”. Silicone blocking may occur due to cross-linking between silicone material of the two engaging portions while the two engaging portions are in contact, which may cause irreversible bonding of the two (or more) engaging portions. For example, with a body having the at least two engaging portions formed from a silicone material, the two engaging portions may fuse together such that the re-closable lumen of the body may not be opened and/or adjusted for implantation. This may occur during packaging, storage, and/or transportation of the IMD for implantation in the patient. If the lumen is fused together and cannot be opened, the IMD and/or at least the operative element may be un-usable. However, examples are not so limited and in some examples, the body may be designed for latter fusing together the two engaging portions while implanted.

FIGS. 14A-14C are flow diagrams, which may comprise part of and/or are example implementations of a flow diagram in example methods (e.g., method 200). Forming the body, as shown at 212 in FIG. 14A, may comprises forming at least one arm comprising a proximal portion that extends from a base of the body and a distal portion opposite the proximal portion. In some examples, the engaging portions of the body comprise at least one surface of the at least one arm, wherein the base and the at least one arm at least partially define the re-closable lumen that at least partially encircles a nerve. In some examples, the at least one arm may comprise a length sufficient to at least partially encircle a nerve and to at least partially overlap or otherwise contact the other of the engaging portions. In some examples, at least two of the engaging portions comprise overlapping portions which may overlap one another to at least partially encircle the nerve and/or form the re-closable lumen.

In some examples, forming the body may comprise forming at least a first arm and a second arm, and/or may comprise forming a first arm, a second arm, and a third arm, as further illustrated by FIGS. 15, 16A-16B, and 17A-17C. In such examples, the engaging portions of the body may comprise at least one surface of the first arm, the second arm, and/or the third arm.

As shown at 220 in FIG. 14B, the method, such as the method 200 in FIG. 13, may further comprise forming a surface-contact feature on a surface of at least one of the engaging portions, wherein the surface-contact feature impacts a surface contact area between the engaging portions. In some examples, the surface may comprise an inner surface, an outer surface, an abutting end, and/or a side surface of the at least one arm, as shown at 222 in FIG. 14C.

FIGS. 15-19 are diagrams schematically illustrating different example devices comprising a body formed at least partially from a silicone material. In some examples, the devices comprise electrode cuffs that comprise at least one contact electrode. However, examples are not limited to those illustrated herein. In some examples, the electrode cuffs illustrated by FIGS. 15-19 may be formed by the method 200 illustrated by FIG. 13. Each of the electrode cuffs illustrated by FIGS. 15-19 comprises at least one arm and engaging portions, as described above in connection with FIG. 14A, with at least one of the engaging portions being formed from the silicone material. In some examples, each of the engaging portions are formed from the silicone material and, in some examples, the entire electrode cuff and/or cuff body is formed from the silicone material. The at least one arm is at least partially self-wrappable about a nerve of a patient and at least partially defines a re-closable lumen. The arm may thereby have a nerve-contacting surface and a non-nerve contacting surface.

Maintaining independence of the engaging portions, at least prior to implantation, may allow for properly implanting the IMD within the patient. In some examples, direct contact between silicone material of the engaging portions may result in the engaging portions fusing together, which may hinder implantation and/or self-sizing of the electrode cuff. In some examples, to prevent or mitigate the engaging portions from fusing together (e.g., cross-linking of the silicone material of engaging portions) prior to being implanted in the patient and/or during implantation of the IMD system, at least one surface of the engaging portion may comprise a surface-contact feature. The surface with the surface-contact feature may be the non-nerve contacting surface, the nerve-contacting surface, a shelf formed at a junction of the proximal portion and the distal portion of the arm, and/or a surface of the base of the body, among other surfaces, such as an abutting end of the arm. In some examples, the surface-contact feature may be a sufficient size to prevent or mitigate cross-linking between the silicone material of the engaging portions during packaging, transportation, storage and/or implantation of the body in a patient, and allows for contact between the engaging portions while the body is implanted in the patient. By preventing cross-linking (e.g., fusing together), independence of the engaging portions may maintained.

FIG. 15 is a diagram schematically illustrating an example device comprising an operative element. In some examples, the operative element may comprise an electrode cuff 300 comprising a cuff body 301, which in some examples may be implemented as the operative element 60 in lead system 51 of FIG. 3B and/or the operative element 60 in the IMD 80 of FIG. 4. In some examples, as illustrated by FIG. 15, the electrode cuff 300 may be coupled to a lead body 50.

As illustrated in FIG. 15, the electrode cuff 300 may comprise a cuff body 301 and at least one contact electrode, such as the array 302 of electrodes 303-1, 303-2, 303-3. In some examples, the cuff body 301 defines a lumen 340 through which a target nerve or other body structure may extend. Among other features, the cuff body 301 may comprise a pair of arms 334, 349 (e.g., flange members) that have a generally arcuate shape and that extend from a base 320 of the cuff body 301. By pulling the ends of the resiliently, in some examples, biased arms 334, 349 apart from each other, access to lumen 340 is provided for engaging a target nerve. Upon release of the arms 334, 349, the cuff body 301 may resume the shape illustrated in FIG. 15. As will be further described, once positioned on a nerve, the materials and construction of the arms 334, 349 permit automatic expansion of the size of lumen 340 to accommodate expansion of the size of the nerve encircled by the cuff body 301. In this manner, the electrodes 303-1, 303-2, 303-3 are held in close contact against the nerve while allowing for expansion of a diameter of lumen 340 defined the arms 334, 349 and the base 320.

In some examples, electrodes 303-1, 303-2, 303-3 are embedded within a wall of the cuff body 301 with the respective electrodes 303-1, 303-2, 303-3 spaced apart from each other along a length of the cuff body 301. In some examples, the electrodes 303-1, 303-2, 303-3 are aligned in series along a single longitudinal axis on a common side or portion of the cuff body 301.

In various examples, as illustrated by FIGS. 17A-17C, the electrode cuff 300 additionally comprises an outer (third) arm 370 that is biased and configured to maintain releasable coverage of at least a portion of an outer surface of the cuff body 301 and of a re-closable opening 309 between the distal portions of arms 334, 349. As illustrated by FIGS. 17A-17C, the outer arm 370 may overlap the first and second arms 334, 349. However, in some examples, this outer arm 370 is omitted.

In some examples, to implant the electrode cuff 300, independence of the at least two engaging portions of the electrode cuff 300 may be maintained. The at least two engaging portions may comprise at least one surface of the first arm 334 and the second arm 349, such as abutting ends, inner surfaces, and/or outer surfaces of the first and second arms 334, 349. At least one of the two engaging portions may be formed of a silicone material, and to maintain independence (or at least temporarily prevent the engaging portions from fusing together due to silicone blocking) of the two engaging portions, at least one of the surfaces of the two engaging portions may comprise a surface-contact feature, as further described herein.

In some non-limiting examples, the body 301 and/or electrode cuff 300 may comprise at least some of substantially the same features and attributes as described within at least U.S. Patent Publication No. 2011/0160827, published on Jun. 30, 2011 and entitled “ELECTRODE LEAD SYSTEM”, U.S. Pat. No. 9,227,055 issued Jan. 5, 2016 and entitled “SELF EXPANDING ELECTRODE CUFF”, and/or U.S. Pat. No. 8,340,785 issued Dec. 25, 2012 and entitled “SELF EXPANDING ELECTRODE CUFF”, the entire teachings of which are incorporated herein by reference in their entireties.

FIGS. 16A-16B are diagrams schematically representing example device comprising an electrode cuff 400. FIG. 16A is an end view of an expandable electrode cuff 400 comprising a cuff body 410. In various examples, the cuff body 410 of the expandable electrode cuff 400 is a single, unitary molded body piece that comprises a base 420, a first arm 434, and a second arm 449. In some examples, the expandable electrode cuff 400 may be implemented as the operative element 60 in lead system 51 of FIG. 3B and/or the operative element 60 in the IMD 80 of FIG. 4. The base 420 has a top wall 422 and a bottom wall 424 extending from a first side wall 426 to a second side wall 428. Each of the first arm 434 and the second arm 449 comprise a proximal portion 431, 435 that extends from the base 420 of the body outwardly and away from the other respective proximal portion 431, 435 and a distal portion 432, 436 that extends toward the other respective distal portion 432, 436.

More specifically, the first arm 434 extends from the proximal portion 431 to the distal portion 432, and is located at the top wall 422 of the base 420 to extend from the first side wall 426 to the distal portion 432. A second arm 449 extends from the proximal portion 435, and is located at the top wall 422 of the base 420 to extend from the second side wall 428 to the distal portion 436. As further described below, the electrode cuff 400 is expandable both during implantation of the electrode cuff 400, and after the electrode cuff 400 is positioned around a desired nerve for delivery of electrical stimulation therapy to the nerve via the engaging portions of the electrode cuff 400. As further illustrated herein, the engaging portions of the electrode cuff 400 (e.g., the first arm 434 and second arm 449) are overlapping portions which may overlap one another to define the re-closable lumen 440.

During the normal, unbiased state of the electrode cuff 400, prior to insertion around the nerve, the electrode cuff 400 is in a fully engaged position, shown in FIG. 16A, in which the distal portion 432 of the first arm 434 is positioned adjacent to and may engage against the second side wall 428 at a location below the proximal portion 435 of the second arm 449 and below the top wall 422 of the base 420. In some examples, the distal portion 436 of the second arm 449 is positioned at a location above the proximal portion 431 of the first arm 434 and above the top wall 422 of the base 420 along the first side wall 426.

The first arm 434 has a length greater than the second arm 449 so that when the electrode cuff 400 is in the fully engaged position, the first arm 434 and the second arm 449 form a lumen 440 for receiving a nerve therein, with an inner surface 441 (e.g., inner side wall) of the second arm 449 forming an inner wall 442 of the lumen 440 so as to position the electrodes (shown in FIG. 15), which may be embedded within the second arm 449, adjacent to the nerve (not shown in FIG. 16A).

When the electrode cuff 400 is in the fully engaged position, an inner surface 444 (e.g., inner side wall) of the first arm 434 is overlapping with (e.g., is positioned over and engages against) an outer surface 446 (e.g., an outer side wall) of the second arm 449, and therefore an outer surface 448 of the first arm 434 forms an outer wall 443 of the lumen 440. In this way, when the electrode cuff 400 is in the fully engaged position shown in FIG. 16A, both the first arm 434 and the second arm 449 extend over the base 420, the first arm 434 forms an outer portion of the electrode cuff 400 (e.g., outer wall 443), and the second arm 449 forms an inner portion of the electrode cuff 400 (e.g., the inner wall 442) for engaging the nerve. Depending on the size of the nerve, once positioned about a nerve, the electrode cuff 400 may be in either the fully engaged position of FIG. 16A or in a partially fully engaged position, wherein the distal portion 432 of the first arm 434 may be spaced from rather than engaged against the second side wall 428, and the distal portion 436 of the second arm 449 may be spaced from rather than aligned with the first side wall 426 at the top wall 422 of the base 420.

In order to implant the electrode cuff 400, in some examples, independence of the at least two engaging (e.g., overlapping) portions of the cuff body 410 may be maintained, at least until the electrode cuff 400 is implanted. In some examples, as noted above, at least two engaging portions comprise overlapping portions, which may comprise at least one surface of the first arm 434 and the second arm 449, wherein the base 420 and at least one of the first arm 434 and the second arm 449 at least partially define the re-closable lumen 440. In some examples, at least one of the at least two overlapping portions is formed of a silicone material, and to prevent (or at least temporarily prevent) silicone blocking, the at least one surface may comprise a surface-contact feature, as further described herein. The at least one surface may comprise the inner surface 444 of the first arm 434, the outer surface 448 of the first arm 434, the inner surface 441 of the second arm 449, the outer surface 446 of the second arm 449, an abutting end of the first arm 434 (e.g., at or near reference 432), an abutting end of the second arm 449 (e.g., at or near reference 436), a side surface of the first arm 434, and/or a side surface of the second arm 449 and/or a surface of the first side wall 426 and/or second side wall 428 of the base 420. In some examples, each of the base 420, the first arm 434, and the second arm 449 may be formed from the silicone material.

FIG. 16B is an end view of the expandable electrode cuff 400 of FIG. 16A in an intermediate open position. As illustrated in FIG. 16B, during positioning of the electrode cuff 400 over the desired nerve, the electrode cuff 400 is advanced from the fully engaged position shown in FIG. 16A, to an intermediate position shown in FIG. 16B, in which the distal portion 432 of the first arm 434 is advanced away from the second side wall 428, and the inner surface 444 of the first arm 434 is advanced away from the outer surface 446 (e.g., outer side wall) of the second arm 449 so that the distal portion 432 extends outward from and along the first side wall 426 of the base 420. When in the intermediate position, the at least two overlapping portions may no longer be overlapping. For example, when the electrode cuff 400 is in the intermediate open position, the first arm 434 may not extend over the top wall 422 of the base 420, and the distal portion 432 may no longer be positioned below the top wall 422 of the base 420, while the second arm 449 may not extend over the top wall 422 of the base 420. In addition, the inner surface 444 of the first arm 434 may no longer be positioned over the outer surface 446 of the second arm 449.

FIGS. 17A-17C are diagrams schematically representing an example device comprising an electrode cuff 305. In some examples, the electrode cuff 305 and/or cuff body 311 illustrated by FIGS. 17A-17C may comprise at least some of substantially the same features and attributes as the electrode cuff 300 and/or cuff body 301 as previously described in association with at least FIG. 15. In some examples, electrode cuff 305 may be implemented as the operative element 60 in lead system 51 of FIG. 3B and/or the operative element 60 in the IMD 80 of FIG. 4. As illustrated in FIG. 17A, according to various examples, an expandable electrode cuff 300 comprises a cuff body 311 that includes a base 320 having a top wall 322 and a bottom wall 324 extending from a first side wall 326 to a second side wall 328, a first arm 334, a second arm 349, and a third arm 370. In such examples, the third arm 370 has a first end secured to an outer surface 347 of the first arm 334 and extending from the first end to overlap, and be in releasable contact with, a portion of the second arm 349 and the first arm 334. The third arm 370 terminates in a second end at a location over the second arm 349. A lumen 340 may be defined by the engaging portions of the first arm 334 and the second arm 349, with a substantially re-closable opening defined by engaging portions (e.g., abutting ends) of the first arm 334 and the second arm 349. In some examples, at least some of the engaging portions may comprise overlapping portions. The second end of the third arm 370 is located generally opposite the substantially re-closable opening of the body.

In some examples, the third arm 370 is bonded at a proximal portion 331 to the first arm 334 along a portion of an outer surface 347 of the first arm 334, and extends outward and over the top wall 322 of the base 320 from the first side wall 326 to a distal portion 332. The second arm 349 extends outward and over the top wall 322 of the base 320 from the second side wall 328 to a distal portion 336. The first arm 334 extends (from the proximal portion 351) outward at the top wall 322 of the base 320 from the first side wall 326 to a distal portion 352.

In some examples, the base 320, the first arm 334, and the second arm 349 are formed from a single, unitary molded cuff body 311 piece, with the third arm 370 is bonded to the molded piece. As further described below, the electrode cuff 305 is expandable both during implantation of the electrode cuff 305, and after the electrode cuff 305 is positioned around a desired nerve for delivery of electrical stimulation therapy to the nerve, similar to the electrode cuffs 300, 400 described above in the examples of FIGS. 15-16B.

During normal, unbiased state of the electrode cuff 305, prior to insertion around the nerve, the electrode cuff 305 is in a fully engaged position, shown in FIG. 17A, in which the third arm 370 extends outward and over the top wall 322 of the base 320 from the first side wall 326 to the distal portion 332, the second arm 349 extends outward and over the top wall 322 of the base 320 from the second side wall 328 to the distal portion 336, and the first arm 334 extends outward at the top wall 322 of the base 320 from the first side wall 326 to the distal portion 352 so that the first arm 334 does not extend over the top wall 322. In addition, while in the fully engaged position, the distal portion 332 of the third arm 370 is positioned adjacent to and engaged against an outer surface 346 of the second arm 349 and an outer surface 347 of the first arm 334, and the distal portion 336 of the second arm 349 is positioned adjacent to and may be engage against the distal portion 352 of the first arm 334 at a location along the third arm 370.

The third arm 370 has a length greater than the second arm 349 so that when the electrode cuff 305 is in the fully engaged position, the first, second and third arms 334, 349 and 370 form a lumen 340 for receiving a nerve therein, with an inner surface 341 (e.g., inner side wall) of the second arm 349 and an inner surface 354 (e.g., inner side wall) of the first arm 334 forming an inner wall 342 of the lumen 340 so as to position the electrodes (shown in FIG. 17A), which are embedded within the second arm 349, adjacent to the nerve (not shown in FIG. 17A). In addition, when the electrode cuff 305 is in the fully engaged position, an inner surface 344 of the third arm 370 is overlapping with (e.g., positioned over) the outer surface 346 of the second arm 349 and the outer surface 347 of the first arm 334, and an outer surface 348 of the third arm 370 forms an outer wall 343 of the lumen 340. In this way, when the electrode cuff body 311 is in the fully engaged position shown in FIG. 17A, both the third arm 370 and the second arm 349 extend over the base 320, the third arm 370 forms an outer portion of the electrode cuff 305 (e.g., the outer wall 343), and the first and second arms 334, 349 form an inner portion of the electrode cuff 305 (e.g., the inner wall 342) for engaging the nerve. Depending on the size of the nerve, once positioned about the nerve, the electrode cuff 305 may be either in the fully engaged position of FIG. 17A or in a partially fully engaged position, wherein the distal portion 332 of the third arm 370 may be positioned along the outer surface 346 of the second arm 349 to be spaced further away from the second side wall 328 than shown in FIG. 17A, and the distal portion 336 of the second arm 349 may be spaced further away from and not engaged against the distal portion 352 of the first arm 334, as described further below.

To implant the electrode cuff 305, in some examples, independence of the engaging portions of the cuff body 311 may be maintained, at least until the electrode cuff 305 is implanted. At least one of the engaging portions may be formed of a silicone material, and to prevent (or at least temporarily prevent) the engaging portions from fusing together due to silicone blocking, the at least one surface may comprise a surface-contact feature, as further illustrated herein. In such examples, the at least one surface of the engaging portions of the body 311 may comprise a portion of the first arm 334, the second arm 349 and/or the third arm 370. For example, the at least one surface of the engaging portions may comprise an inner surface 344 of the third arm 370 configured to be in releasable contact with the portions of the first arm 334 and the second arm 349 and/or an outer surface 347, 346 of the first arm 334 and/or the second arm 349 configured to be in releasable contact with the portions of the third arm 370. In some examples, the at least one surface additional or alternatively may comprise the abutting ends (e.g., near the numerals 352, 336) of the first arm 334 and/or the second arm 349 and/or the third arm 370.

In various examples, the abutting end of the distal portion 352 of the first arm 334 may define a shelf 353. The shelf 353 may provide an area by which an abutting end of the second arm 349 may make releasable contact with first arm 334. The shelf 353 may comprise an additional engaging portion, which overlaps or abuts with the abutting end of the second arm 349. In some examples, this arrangement limits the extent of rotational movement of the second arm 349, which may bolster structural integrity of cuff body and also defines minimum diameter of the lumen 340 so as to prevent undue pressure and/or constriction on a nerve. In some non-limiting examples, the body 311 and/or electrode cuff 305 may comprise at least some of substantially the same features and attributes as described within at least U.S. Pat. No. 8,340,785, issued Dec. 25, 2012 and entitled “SELF EXPANDING ELECTRODE CUFF”, U.S. Pat. No. 9,227,053, issued Jan. 5, 2016 and entitled “SELF EXPANDING ELECTRODE CUFF”, U.S. Pat. No. 8,934,992, issued Jan. 13, 2015 and entitled “NERVE CUFF”, and/or U.S. Publication No. 2020/0230412, published on Jul. 23, 2020 and entitled “CUFF ELECTRODE”, the entire teachings of each of which are incorporated herein by reference in their entireties.

FIG. 17B is an end view of the example expandable cuff 305 of FIG. 17A in an intermediate open position. As illustrated in FIG. 17B, during positioning of the electrode cuff 305 over the desired nerve so that the nerve may be properly located within the lumen 340, the electrode cuff 305 is advanced from the fully engaged position shown in FIG. 17A, to an intermediate position shown in FIG. 17B in which the distal portion 332 of the third arm 370 is advanced away from the second side wall 328, and the third arm 370 is advanced away from the second arm 349 so that the distal portion 332 extends outward in an opposite direction from the first side wall 326 of the base 320. When the electrode cuff 305 is in the intermediate open position, the engaging portions may not be overlapping and/or may not otherwise be in contact with another engaging portion. For example, in the intermediate open position, the third arm 370 is not overlapping with (e.g., not positioned so as to extend over the top wall 322 of) the base 320, while the second arm 349 may remain positioned to extend over the top wall 322 of the base 320, with the distal portion 336 adjacent to the distal portion 352 of the first arm 334. In addition, the inner surface 344 of the third arm 370 is no longer positioned over and adjacent to the outer surface 346 of the second arm 349 when the electrode cuff 305 is in the intermediate open position of FIG. 17B.

FIG. 17C is an end view of the example expandable electrode cuff 305 of FIG. 17A in a fully open position. As illustrated in FIG. 17C, once the electrode cuff 305 is in the intermediate open position, the distal portion 336 of the second arm 349 is advanced away from the distal portion 352 of the first arm 334 and the second arm 349 is advanced to no longer extend over the top wall 322 of the base 320 and the first side wall 326 so that the distal portion 336 extends outward in the opposite direction from the second side wall 328, resulting in the inner surface 341 of the second arm 349 no longer forming the lumen 340 when the electrode cuff 305 is in the fully open position of FIG. 17C. As a result, when the electrode cuff 305 is in the fully open position, neither the third arm 370 nor the second arm 349 are overlapping and/or positioned so as to extend over or near the top wall 322 of the base 320 of the electrode cuff 305. For example, the inner surface 344 of the third arm 370 is no longer positioned over or near the outer surface 346 of the second arm 349, and the inner surface 341 of the second arm 349 no longer forms the inner wall 342 of the lumen 340.

By mitigating silicone blocking from occurring between engaging portions at least prior to implantation, in accordance with various examples, enables the electrode cuff 305 to be advanced between the fully engaged position, the intermediate open position, and the fully open position, and to position the electrode cuff 305 over a nerve during implantation, as previously described.

In some non-limiting examples, the body 311 and/or electrode cuff 305 may comprise at least some of substantially the same features and attributes as described within at least U.S. Pat. No. 8,340,785, issued Dec. 25, 2012 and entitled “SELF EXPANDING ELECTRODE CUFF”, U.S. Pat. No. 9,227,053, issued Jan. 5, 2016 and entitled “SELF EXPANDING ELECTRODE CUFF”, the entire teachings of which are each incorporated herein by reference in their entireties.

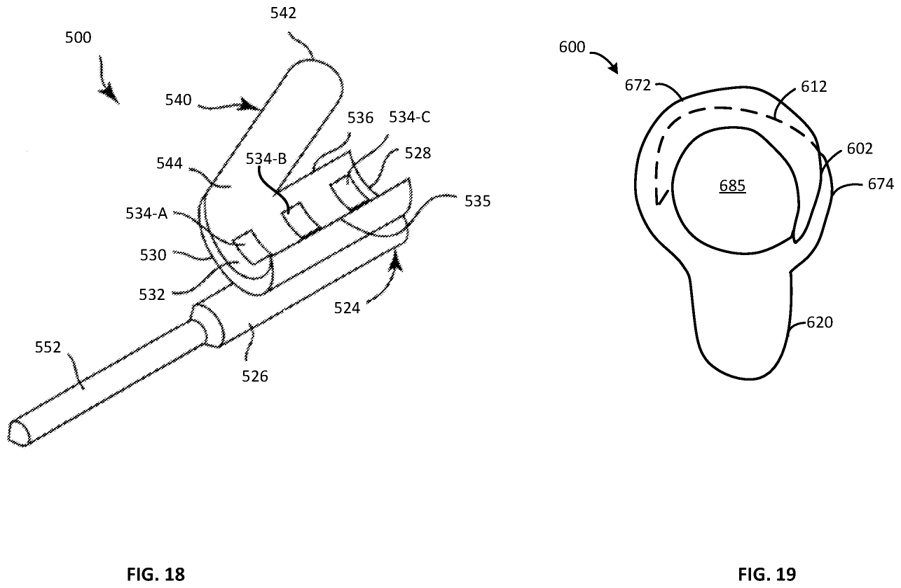

FIG. 18 is a diagram schematically illustrating a device comprising an example electrode cuff 500. In some examples, the electrode cuff 500 comprises a cuff body 532 which may be coupled to a lead comprising a medical lead body 552 and a distal cuff portion 524. In some examples, the electrode cuff 500 may be implemented as the operative element 60 in lead system 51 of FIG. 3B and/or the operative element 60 in the IMD 80 of FIG. 4. In some examples, the distal cuff portion 524 comprises a spine portion 526 that extends from medical lead body 552, and which supports a cuff body 532 defining a first elongate portion. The cuff body 532 comprises a distal end 528 and a proximal end 530 and a series of contact electrodes 534A, 534B, 534C extending therebetween to be spaced apart longitudinally along a length of the cuff body 532. The distal cuff portion 524 may comprise an arm 540, defining a second elongate portion and having a distal portion 542 (e.g., a tip) and a proximal portion 544 that extends from one side edge 536 of cuff body 532 (with the cuff body 532 comprises a second side edge 535). In various examples, the arm 540 extends outwardly from the side edge 536 of cuff body 532 to form a generally acute angle (a) relative to side edge 536 of the cuff body 532. The angle (a) may be selected to orient the arm 540 to be interposed between two adjacent nerve branches and to wrap in contact about cuff body 532. In some examples, the proximal portion 544 of the arm 540 is positioned at a proximal end 530 of cuff body 532 so that distal portion 542 is oriented toward the distal end 528 of cuff body 532.

Although not illustrated, the proximal portion 544 of arm 540 may be attached to the distal end 528 of cuff body 532 so that the arm 540 extends toward the proximal end 530 of cuff body 532 at an angle (a). In other words, the arm 540 may extend in direction opposite that shown in FIG. 18.

As shown, the cuff body comprises at least two engaging portions, which may define a re-closable lumen. In some examples, independence of the at least two engaging portions of the cuff body may be maintained, at least until the electrode cuff is implanted. The at least two engaging portions may be formed of a silicone material, and to prevent (or at least temporarily prevent) silicone blocking, at least one surface of the engaging portions may comprise a surface-contact feature, as further illustrated herein. In such examples illustrated by FIG. 18, the at least one surface of the engaging portions of the cuff body 532 may comprise a portion of the arm 540, and/or a side wall of the cuff body 532.

In some non-limiting examples, the cuff body 532 and/or electrode cuff 500 of FIG. 18 may comprise at least some of substantially the same features and attributes as described within at least U.S. Pat. No. 8,934,992, issued Jan. 13, 2015 and entitled “NERVE CUFF”, the entire teachings of which are incorporated herein by reference in its entirety.

FIG. 19 is a diagram schematically illustrating an example electrode cuff 600. In some examples, the electrode cuff 600 comprises a cuff body 660, with the cuff body 660 comprising a base 620, a first arm 672, and a second arm 674. In some examples, the expandable electrode cuff 600 may be implemented as a single, unitary molded piece that comprises the base 620, the first arm 672, and the second arm 674. The first arm 672 and second arm 674 may extend from opposing sides of the base 620. In some examples, the first and second arms 672, 674 of the electrode cuff 600 are expandable both during implantation of the electrode cuff 600, and after the electrode cuff 600 is positioned around a nerve for delivery of electrical stimulation therapy to the nerve.

Similar to FIG. 17A, in some examples, the first arm and second arm 672, 674 overlap to form the lumen 685 and are wrappable about a nerve. The engaging portions of the first arm and second arm 672, 674 may comprise a side surface 602 of the first arm 672 and a side surface 612 of a second arm 674. At least one of the least two engaging portions may be formed from a silicone material, and to prevent (or at least temporarily prevent) silicone blocking at least until the IMD is implanted, at least one surface of the engaging portions may comprise a surface-contact feature, as further illustrated herein. In the examples illustrated by FIG. 19, the at least one surface of the at least two engaging portions of the body may comprise a portion of the first arm 672 and/or the second arm 674, such as a proximal and/or distal portion of the first and/or second arms (e.g., an abutting end), and/or a side surface 612, 602 of the first and/or second arms 672, 674.

FIGS. 20A-20C are diagrams which may comprise part of and/or are example implementations of a flow diagram in example methods (e.g., method 200 of FIG. 13). More particularly, FIGS. 20A-20C (as well as FIGS. 21A-21C, and 22A-22C) illustrate example methods and/or example devices comprising forming the surface-contact feature(s) on the surface of at least one engaging portion of the operative element, such as an electrode cuff. In some examples, the method illustrated by FIG. 20A and/or the devices illustrated by FIGS. 20B-20C may comprise at least some of substantially the same features and attributes and/or are example implementations of the method as previously described in association with at least FIG. 14B. As shown at 701 in FIG. 20A, forming the surface-contact feature may comprise forming a plurality of indented or protruding features. The plurality of indented or protruding features, in some examples, comprise a smaller size than a surface area of the engaging portions. For example, the plurality of indented or protruding features may be located on twenty-five percent or less of the engaging surface area.

FIGS. 20B-20C illustrate example devices comprising surface-contact features. For example, FIGS. 20B-20C illustrate different example devices comprising a cuff body 702, 703 of an electrode cuff that includes a base 720, a first arm 734, a second arm 749, and a third arm 770. In some examples, the cuff bodies 702, 703 illustrated by FIGS. 20B-20C may comprise at least some of substantially the same features and attributes as the cuff body 311 as previously described in association with at least FIGS. 17A-17C, with the addition of a plurality of protruding features 753 or indented features 755.

The protruding features 753, as shown by FIG. 20B, may comprise bumps located on the cuff and/or an arm to reduce the contact area between the engaging portions. For example, the bumps may be located on an outer surface of the second arm 749, an inner surface of the third arm 770 and/or a surface of the base 720 which the third arm 704 may overlap with. The surface with the protruding features 753 may comprise a non-nerve contacting surface, in some examples, and in other examples, may comprise a nerve-contacting surface. Placing the protruding features 753 (or other surface-contact features) on non-nerve contacting surfaces may mitigate interference with contact between the contract electrodes and the nerve. FIG. 20C illustrates a cuff body similar to FIG. 20B but with a plurality of indented features 755. The indented and/or protruding features 753, 755 are not limited to geometric shapes and/or number of features as illustrated by FIGS. 20B-20C and may comprise a variety of shapes, sizes, and numbers.

FIGS. 21A-21C are diagrams which may comprise part of and/or are example implementations of a flow diagram in an example method (e.g., method 200). In some examples, the method illustrated by FIG. 21A and/or the devices illustrated by FIGS. 21B-21C may comprise at least some of substantially the same features and attributes and/or are example implementations of the method as previously described in association with at least FIG. 14B. In some examples, as shown at 800 in FIG. 21A, forming the surface-contact feature may comprise altering a surface chemistry of the surface of the engaging portions. Altering the surface chemistry may comprise performing a surface treatment, such as a plasma surface treatment. In some examples, the plasma surface treatment comprises performing trimethylsiloxane (TMS) plasma treatment. In some examples, altering the surface chemistry comprising applying a layer of material on the surface, such as a lubricant (e.g., oil), a powder, a dusting (e.g., graphite, talc, tiO2), etc. As an example, altering the surface chemistry may comprise applying a layer of graphite on the surface.

FIGS. 21B-21C illustrate example devices comprising surface-contact features. For example, FIGS. 21B-21C illustrate different example devices comprising a cuff body 805, 807 of an electrode cuff comprising a base 820, a first arm 834, a second arm 849, and a third arm 870. In some examples, the cuff bodies 805, 807 illustrated by FIGS. 21B-21C may comprise at least some of substantially the same features and attributes as the cuff body 311 as previously described in association with at least FIGS. 17A-17C. The cuff body 805 of FIG. 21B further comprises a layer of TMS 821 from a TMS plasma treatment or a spray coated layer of material 823, which may be on an outer surface of the second arm 849, an inner surface of the third arm 870 and/or a surface of the base 820 which the third arm 804 may overlap with. FIG. 21C illustrates a cuff body 807 similar to FIG. 21B but with a spray coated layer of material 823 on surfaces of the second arm 849 and third arm 870 in accordance with various examples.

FIGS. 22A-22C are diagrams which may comprise part of and/or are example implementations of a flow diagram in an example method (e.g., method 200). In some examples, the method illustrated by FIG. 22A and/or the devices illustrated by FIGS. 22B-22C may comprise at least some of substantially the same features and attributes and/or are example implementations of the method as previously described in association with at least FIG. 14B. In some examples, as shown at 900 in FIG. 22A, forming the surface-contact feature may comprise adding texture to the surface of the engaging portions. In some examples, adding the texture may comprise forming protrusions and/or indentations on the surface, such as a plurality of bumps, channels, and/or grooves as illustrated by FIGS. 20B-20C. In some examples, adding the texture comprising roughing a surface of the at least one engaging portion, with the texture comprising an indiscriminate pattern.

FIGS. 22B-22C illustrate example devices comprising surface-contact features. For example, FIGS. 22B-22B illustrate example devices comprising a cuff bodies 909, 911 of an electrode cuff that includes a base 920, a first arm 934, a second arm 949, and a third arm 970. In some examples, the cuff bodies 909, 911 illustrated by FIGS. 22B-22C may comprise at least some of substantially the same features and attributes as the cuff body 311 as previously described in association with at least FIGS. 17A-17C. The cuff body 909 of FIG. 22B further comprises a roughened surface 925 on at least one of the engaging portions. In some examples, both engaging portions comprise a roughed surface. For example, the inner surface of the third arm 974 and the outer surface of the second arm 949 comprise roughed surfaces to reduce surface contact area between the first arm 934 and the second arm 949. FIG. 22C illustrates a cuff body similar to FIG. 22B but with roughened surface being located on a protruding component that extends from the surface of the at least one engaging portions, in accordance with various examples. In some examples, the second arm 949 may comprise protruding components that house at least one contact electrode 927-1, 927-2. In some examples, an IMD system and/or electrode cuff may comprise protruding components at least some of substantially the same features and attributes as described within at least U.S. Publication No. 2020/0230412, published on Jul. 23, 2020 and entitled “CUFF ELECTRODE, the entire teachings of which are incorporated herein by reference in its entirety.

Examples are not limited to bodies and/or cuff bodies comprising contact-surface features. In some examples, forming the body may comprise forming the at least one engaging portion from a self-lubricating silicone material. In some examples, each of the engaging portions are formed from the self-lubricating silicone material. In some examples, forming the body may comprise forming the at least one engaging portion from the silicone material and forming the other of the engaging portions from a different elastomer material. The different elastomer material may not cross-link with the silicone material. Example elastomer materials include polyurethane, PEEK, a silicone-polyurethane blend, ETFE, and polysulfone. In some examples, the base, the first arm, and the second arm may be formed from the silicone material as a unitary component, and the third arm is formed from the different elastomer material, however examples are not so limited. In various examples, the at least one engaging portion may comprise a layer of eluting non-thermoset material. As further described below, examples are not so limited and may comprise a number of variations.

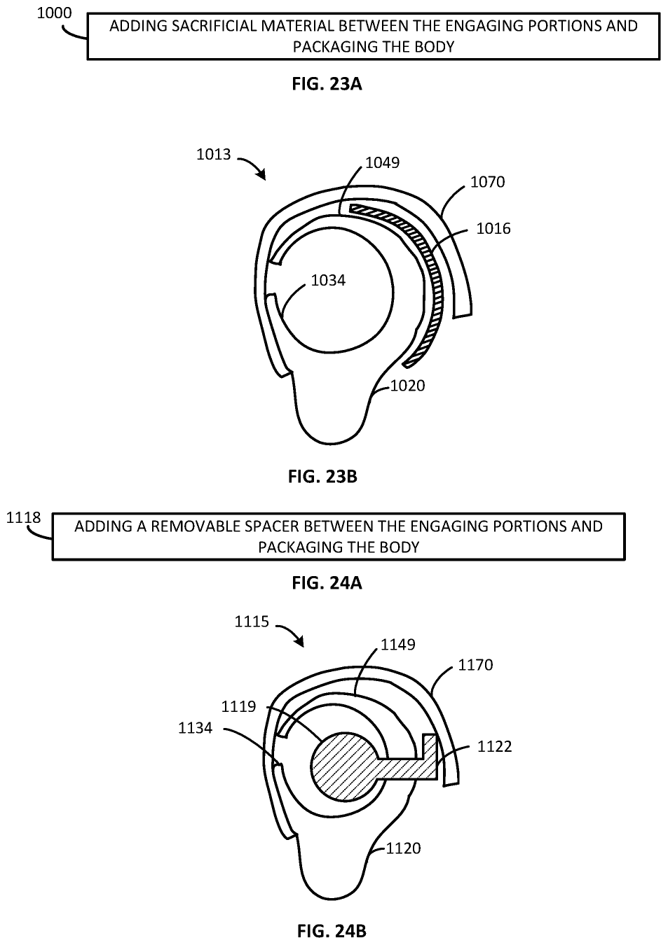

FIGS. 23A-23B are diagrams which may comprise part of and/or are example implementations of a flow diagram in an example method (e.g., method 200). In some examples, the method illustrated by FIG. 23A and/or the device illustrated by FIG. 23B may comprise at least some of substantially the same features and attributes and/or are example implementations of the method 200 as previously described in association with at least FIG. 13. In some examples, as shown at 1000 in FIG. 23A, forming the body comprises adding a sacrificial material between the engaging portions, and packaging the body with the sacrificial material.

FIG. 23B illustrates an example of a device comprising an engaging portion including a sacrificial material. More specifically, FIG. 23B illustrates an example device comprising a cuff body 1013 of an electrode cuff that includes a base 1020, a first arm 1034, a second arm 1049, and a third arm 1070. In some examples, the cuff body 1013 illustrated by FIG. 23B may comprise at least some of substantially the same features and attributes as the cuff body 311 as previously described in association with at least FIGS. 17A-17C, with the addition of the sacrificial material 1016 between the second arm 1049 and the third arm 1070. The sacrificial material 1016 may comprise a disposable or permanent tab formed from a non-silicone material.

FIGS. 24A-24B are diagrams which may comprise part of and/or are example implementations of a flow diagram in an example method (e.g., method 200). In some examples, the method illustrated by FIG. 24A and/or the device illustrated by FIG. 24B may comprise at least some of substantially the same features and attributes and/or are example implementations of the method 200 as previously described in association with at least FIG. 13. In some examples, as shown at 1118 in FIG. 24A, forming the body may comprise forming a removable spacer between the engaging portions, and packaging the body with the removable spacer.

FIG. 24B illustrates an example a device comprising a removable spacer between two or more engaging portions of the device. More specifically, FIG. 24B illustrates an example device comprising a cuff body 1115 of an electrode cuff that includes a base 1120, a first arm 1134, a second arm 1149, and a third arm 1170. In some examples, the cuff body 1115 illustrated by FIG. 24B may comprise at least some of substantially the same features and attributes as the cuff body 311 as previously described in association with at least FIGS. 17A-17C, with the addition of the removable spacer 1119 between the second arm 1149 and the third arm 1170. In some examples, the removable spacer 1119 may comprise a support 1123 located proximal to the inner surface of the lumen formed by the first arm 1134 and the second arm 1149. The removable spacer may further comprise an arm 1122 that wraps between the engaging portions, such as the outer surface of the second arm 1149 and the inner surface of the third arm 1170.

Although the example surface-contact features illustrated by FIGS. 20A-24B illustrate a cuff body comprising three arms, examples are not so limited and may comprise various different operative elements including different configured cuff bodies. In some examples, the electrode cuff and/or cuff bodies illustrated by FIGS. 20A-24B may comprise at least some of substantially the same features and attributes as the electrode cuff 300, 400, 500, 600 as previously described in association with at least FIGS. 15, 16A-16B, 18, and 19.

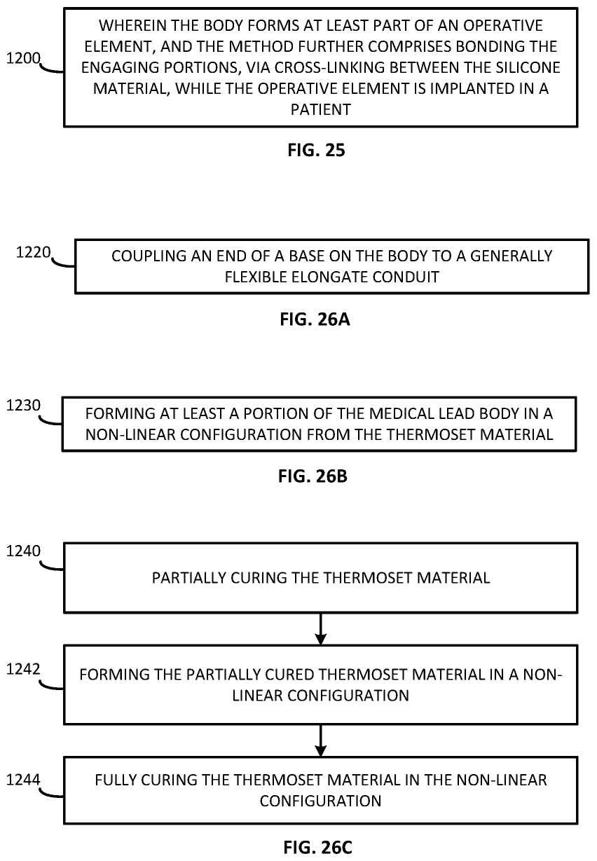

Further, although the above examples describe mitigating cross-linking, examples are not so limited. FIG. 25 is a diagram which may comprise part of and/or is an example implementation of a flow diagram in an example method (e.g., method 200). As shown at 1200 in FIG. 25, the body may form at least part of an operative element, and the method of forming the body may further comprise bonding the engaging portions, via cross-linking between the silicone material, while the operative element is implanted in a patient.

FIGS. 26A-26C are diagrams which may comprise part of and/or are example implementations of a flow diagram in an example method (e.g., method 200). As shown at 1220 in FIG. 26A, the method of forming the body may further comprise coupling a proximal end of a base of the body to a generally flexible elongate conduit. The generally flexible elongate conduit may comprise a medical lead body that carries electrically conductive wires. As an example, the method may further comprise forming at least one lead portion of the lead body in a non-linear configuration from a thermoset material, as shown at 1230 in FIG. 26B. The at least one lead portion may be formed in a variety of ways, as previously described above in connection with at least FIGS. 6-11. In some examples, the lead body and/or the at least one lead portion may be implemented and/or comprise at least some of substantially the same features and attributes of the lead body 50 as previously described in association with at least FIG. 4. As a non-limited example, illustrated by FIG. 26C, forming the at least one lead portion may comprise partially curing the thermoset material, at 1240, forming the partially cured thermoset material in the non-linear configuration, at 1242, and fully curing the thermoset material in the non-linear configuration to form the at least one lead portion, at 1244, however, the examples are not so limited.

Various examples are implemented in accordance with the underlying Provisional Application Ser. No. 63/145,705, entitled “IMPLANTABLE MEDICAL DEVICES AT LEAST PARTIALLY FORMED FROM A THERMOSET MATERIAL,” filed Feb. 4, 2021, to which benefit is claimed and which is fully incorporated herein by reference for its general and specific teachings. For instance, examples herein and/or in the Provisional Application can be combined in varying degrees (including wholly). Reference can also be made to the experimental teachings and underlying references provided in the underlying Provisional Application. Examples discussed in the Provisional Application are not intended, in any way, to be limiting to the overall technical disclosure, or to any part of the claimed disclosure unless specifically noted.

Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein.

1. A method, comprising:
forming at least one lead portion of a medical lead body in a non-linear configuration from a thermoset material.

2. The method of claim 1, wherein forming the at least one lead portion comprises:
forming the thermoset material in a first configuration; and
forming a thermoplastic material on the thermoset material in the first configuration to form the at least one lead portion in the non-linear configuration.

3. The method of claim 2, wherein the first configuration comprises a linear configuration, and forming the thermoset material comprises at least one of extruding and molding the thermoset material in the linear configuration.

4. The method of claim 3, wherein forming the thermoplastic material comprises:
adding a layer of the thermoplastic material on at least one of an inner circumference and an outer circumference of the thermoset material in the linear configuration for the at least one lead portion; and
forming the layer of the thermoplastic material and the thermoset material in the non-linear configuration for the at least one lead portion.

5. The method of claim 1, wherein forming the at least one lead portion comprises:
partially curing the thermoset material;
forming the partially cured thermoset material in the non-linear configuration; and
fully curing the thermoset material in the non-linear configuration to form the at least one lead portion.

6. The method of claim 5, wherein forming the partially cured thermoset material in the non-linear configuration comprises moving an extruding head of an extruder relative to the forming plate or moving the forming plate relative to the extruding head via control circuitry to form the partially cured thermoset material of the at least one lead portion in the non-linear configuration.

7. The method of claim 1, wherein forming the at least one lead portion comprises:
adding a layer of the thermoset material on a thermoplastic material in a linear configuration to form the at least one lead portion in the non-linear configuration.

8. The method of claim 1, wherein forming the at least one lead portion comprises forming a first lead portion in a first configuration extending from a second lead portion in the non-linear configuration from the thermoset material, wherein the non-linear configuration comprises at least one of:
a generally sigmoid pattern, a generally helix pattern, a non-continuous sigmoid pattern, a triangular step function pattern, and a square wave pattern.

9. The method of claim 1, wherein forming the at least one lead portion comprises:
forming a thermoplastic material in the non-linear configuration; and
adding a layer of the thermoset material on the thermoplastic material in the non-linear configuration to form the at least one lead portion.

10. The method of claim 1, the method further comprising coupling an end of the medical lead body to an operative element comprising at least one contact electrode, the operative element comprising engaging portions and at least one of the engaging portions being formed from silicone and the method further comprising:
forming a surface-contact feature on a surface of at least one of the engaging portions, wherein the surface-contact feature reduces the surface contact area between the engaging portions and mitigates silicone blocking, the surface-contact feature comprising a plurality of indented or protruding features of a smaller size than a surface area of the engaging portions.

11. A method, comprising:
forming a body supporting at least one contact electrode, the body at least partially defining a re-closable lumen and comprising engaging portions, at least one of the engaging portions being formed from a silicone material.

12. The method of claim 11, the method further comprising forming a surface-contact feature on a surface of at least one of the engaging portions, wherein the surface-contact feature impacts a surface contact area between the engaging portions.

13. The method of claim 12, wherein the surface-contact feature reduces the surface contact area between the engaging portions and mitigates silicone blocking, the surface-contact feature comprising a plurality of indented or protruding features of a smaller size than a surface area of the engaging portions.

14. The method of claim 12, wherein forming the surface-contact feature comprises performing at least one of:
altering a surface chemistry of the surface of the at least one engaging portion; and
adding texture to the surface of the at least one engaging portion.

15. The method of claim 14, wherein altering the surface chemistry comprises at least one of:
performing a trimethylsiloxane (TMS) plasma surface treatment;
applying a layer of lubricant on the surface; and
applying a layer of graphite on the surface.

16. The method of claim 14, wherein adding texture comprises forming at least one of:
a plurality of protrusions;
a plurality of indentations; and
a roughened surface.

17. The method of claim 11, the method further comprising adding a sacrificial material between the engaging portions, and packaging the body with the sacrificial material between the engaging portions.

18. The method of claim 11, wherein forming the body comprises forming at least one arm comprising a proximal portion that extends from a base of the body and a distal portion opposite the proximal portion, the engaging portions comprising at least one surface of the at least one arm, wherein the base and the at least one arm at least partially define the re-closable lumen that at least partially encircles a nerve.

19. The method of claim 11, wherein the body forms at least part of an operative element, the method further comprises bonding the engaging portions, via cross-linking between the silicone material, while the operative element is implanted in a patient.

20. The method of claim 11, further comprising:
forming at least one lead portion of a medical lead body in a non-linear configuration from a thermoset material; and
coupling a proximal end of a base of the body to the medical lead body.

21-140. (canceled)
